(19) United States
(12) Patent Application Publication
(10) Pub. No.: US 2025/0352259 A1
(43) Pub. Date: Nov. 20, 2025

(54) ENDOVENOUS DEVICE FOR LOCALIZED ABLATION

(71) Applicant: Montra Medical LLC, Oakland, CA (US)

(72) Inventors: Anand Doraiswamy, Dallas, TX (US); Jarren Baldwin, Dallas, TX (US); Joshua Liu, Newark, CA (US)

(21) Appl. No.: 19/209,637

(22) Filed: May 15, 2025

Related U.S. Application Data
(60) Provisional application No. 63/649,255, filed on May 17, 2024.

(51) Int. Cl. A61B 18/10; A61B 18/00

(52) U.S. Cl. CPC A61B 18/10; A61B 2018/00077; A61B 2018/00184; A61B 2018/00214; A61B 2018/00404; A61B 2018/00642; A61B 2018/00702; A61B 2018/00761; A61B 2018/00791; A61B 2018/0091

(57) ABSTRACT

This disclosure relates to endovenous devices and methods for treating varicose veins. The endovenous devices can include a heating element that is heated with resistive heating from direct current flowing therethrough. The direct current can be provided by a battery disposed in a handheld controller that can modulate the direct current based on sensed temperatures proximate the heating element and clinician commands. The heating element can include self-expanding features to bring the heating element in contact with an inner wall of a vein to facilitate heat transfer and mechanically agitate the inner wall of the vein to occlude the vein.

### CROSS REFERENCE TO RELATED APPLICATIONS

This application claims priority to U.S. Provisional Patent Application No. 63/649,255, filed May 17, 2024, which is incorporated herein by reference in its entirety. Any and all applications, if any, for which a foreign or domestic priority claim is identified in the Application Data Sheet of the present application are hereby incorporated by reference under 37 CFR 1.57.

### FIELD

This disclosure relates to endovenous devices for localized ablation, specifically to endovenous devices to treat chronic venous disease.

### BACKGROUND

The vascular system carries blood throughout the body. The vascular system includes arteries that distribute blood containing oxygen from the heart throughout the body and veins that carry deoxygenated blood back to the heart. Veins close to the surface of the skin of a patient, sometimes referred to as superficial veins, can become varicose. Varicose veins can sometimes be seen on the exterior of the patient as bulging, enlarged, and/or twisted veins. In some instances, varicose veins are not simply cosmetic and can cause pain, discomfort, and/or other health problems. Varicose veins can be found in different regions of the human body but are often found to affect the veins in the legs and cause chronic venous disease.

### SUMMARY

Varicose veins can be treated with radiofrequency ablation (RFA). A radiofrequency ablation (RFA) catheter can be navigated through the vasculature to a varicose vein. A radiofrequency generator can supply radiofrequency energy (i.e., alternating electrical current) to the tip of the RFA catheter. The radiofrequency energy heats the tip to apply heat to the varicose vein. Heat from the RFA catheter can damage (e.g., scar) the inside wall of the vein, which can cause the vein wall to collapse to occlude the vein and impede blood flow to the vein. External compression can be applied to the patient's skin during RFA to help the tip of the RFA catheter contact the vein wall to cause damage. After heat is applied, the RFA catheter can be removed and, over time, the treated vein can shrink and eventually be absorbed by the body.

RFA treatment of varicose veins has numerous drawbacks. For example, RFA generators require significant power, and can be expensive and cumbersome. The generators are typically tabletop devices that are not easily handled. Additionally, applying external compression to a patient can be uncomfortable and provide unreliable contact between the vein wall and the tip of the RFA catheter, which can result in multiple passes with the RFA catheter being used to occlude the vein.

The endovenous devices disclosed herein can at least address one or of the foregoing drawbacks. For example, the endovenous devices can be handheld devices that are easily carried by a clinician. The endovenous devices can be battery powered. The endovenous devices can be low cost, which can enable the devices to be disposable in some variants. The battery of the endovenous devices can be rechargeable in some variants. The endovenous devices can include a self-expanding tip that can expand to contact the inner wall of the vein which, in some instances, may eliminate the need for external compression and/or multiple passes. The self-expanding tip can better damage the inner wall (e.g., endothelium) of the vein to cause occlusion. The self-expanding tip can include mechanical or abrasive features to disrupt the inner wall of the vein.

The endovenous devices described herein can utilize resistive heating, which can also be described as Joule heating and/or ohmic heating, to apply heat to the inner wall of a vein to treat varicose veins. The endovenous devices can utilize direct current to heat a heating element, which can be expandable and/or self-expanding in some variants. The use of direct current can enable the endovenous devices to operate with a battery, as opposed to a generator as used with RFA. The endovenous devices can include a first conductor (e.g., positive conductor) that can conduct direct current from a power source (e.g., battery, power connection) to the heated element and a second conductor (e.g., negative conductor) that can conduct direct current away from the heated element. The first and second conductors can include the same or different material and/or the same or different cross-section size and/or shape. The first and second conductors can be insulated. The direct current flowing through the heated element can heat the heated element with resistive heating. The resistance of the heated element can be increased relative to the first and/or second conductors to produce more heat at the heated element than at the first and/or second conductors by including a material with higher resistance (e.g., Nitinol) and/or including a geometry with a higher resistance (e.g., smaller cross-section) at the heated element. The controller of the endovenous devices can adjust the direct current, which can include providing modulated variable current (e.g., direct current) and/or pulse-width modulation. The endovenous devices can include one or more thermocouples to monitor a temperature at the heated element and/or other locations, and the controller can adjust the current based on the sensed temperature. The heat from the heating element can damage the inner wall of the vein to cause occlusion. In some variants, the endovenous devices herein can utilize alternating current, radio frequency, and/or non-radio frequency to ablate.

The endovenous devices described herein can include expandable features (e.g., self-expanding features) to contact and damage the inner wall of the vein. In some variants, the heated element can include the expandable (e.g., self-expanding) features to contact the heated element with the inner wall of the vein to promote heat transfer to damage the inner wall. In some variants, the heated element and the expandable (e.g., self-expanding) features can be separately operated such that the expandable (e.g., self-expanding) features can contact the inner wall separate from the heated element. The expandable (e.g., self-expanding) features can mechanically damage the inner wall (e.g., endothelium) of the vein. The expandable (e.g., self-expanding) features can include one or more expanding (e.g., self-expanding) wires, loops, coils, hooks, strips, rings, and/or other features. The expandable (e.g., self-expanding) features can include a shape memory material, such as a nickel titanium alloy (e.g., Nitinol). The expandable (e.g., self-expanding) features can include abrasive features (e.g., textures, edges, points, etc.) to damage the inner wall of the vein. The expandable (e.g., self-expanding) features can oscillate at various frequencies, vibrate, rotate, and/or include other movements to disrupt the endothelium, which can include with or without the heated element being heated.

The endovenous devices can include irrigation and/or aspiration functionality. In some variants, irrigation and/or aspiration devices can be used in conjunction with the endovenous devices described herein. The endovenous devices can be used in conjunction with the delivery of fluids, agents (e.g., local anesthetic such as lidocaine), sclerosants, glue, therapeutics, medicaments, nutrients, etc. In some variants, the combination of the expandable (e.g., self-expanding) features and the heated element can enable lower temperatures to be used in comparison to the heated element alone.

Neither the preceding summary nor the following detailed description purports to limit or define the scope of protection. The scope of protection is defined by the claims. Furthermore, reference is made herein to removing thrombi from veins and/or plaque from blood vessels, such as arteries. One of ordinary skill in the art will understand, after reviewing the entirety of this disclosure, that the systems and methods described herein may be applied to removing other occlusions from blood vessels of the body.

### BRIEF DESCRIPTION OF THE DRAWINGS

The abovementioned and other features of the embodiments disclosed herein are described below with reference to the drawings of the embodiments. The illustrated embodiments are intended to illustrate, but not to limit, the scope of protection. Various features of the different disclosed embodiments can be combined to form further embodiments, which are part of this disclosure.

FIG. 1A illustrates the venous system of the lower extremities.

FIG. 1B illustrates a limb with varicose veins.

FIG. 2A illustrates an ablation assembly.

FIG. 2B illustrates a controller.

FIG. 2C illustrates an elongate element of the ablation assembly.

FIG. 2D illustrates an ablation assembly with an actuator.

FIG. 2E illustrates the actuator of the ablation assembly deploying an elongate element from a sheath.

FIG. 3A illustrates a limb with varicose veins.

FIG. 3B illustrates an endovenous device with an elongate element disposed through the vasculature to place a heated element within a varicose vein.

FIG. 3C shows an enlarged view of the heated element of FIG. 3B within the varicose vein.

FIG. 4 illustrates a loop of an elongate element with a plurality of heated elements.

FIG. 5A illustrates a coiled heated element of an elongate element.

FIG. 5B illustrates another view of the coiled heated element of FIG. 5A.

FIG. 5C illustrates another view of the coiled heated element of FIG. 5A.

FIG. 5D illustrates another view of the coiled heated element of FIG. 5A in a vein.

FIG. 6A illustrates a tapered-coil heated element in a vein.

FIG. 6B illustrates a tapered-coil heated element in a vein.

FIG. 7A illustrates first and second conductors and a heating element.

FIG. 7B illustrates first and second conductors and an elongate heating element.

FIG. 8 illustrates an elongate element with a ring heating element.

FIG. 9 illustrates an elongate element with a coil heating element.

FIG. 10 illustrates an elongate element with a plurality of heated element strips.

FIG. 11 illustrates an elongate element with a plurality of heated element strips being expanded.

FIG. 12A illustrates an elongate element with a steerable tip.

FIG. 12B illustrates the elongate element of FIG. 12A being navigated through vasculature.

FIG. 13 illustrates an elongate element disposed within a sheath.

FIG. 14 illustrates an elongate element disposed within a sheath.

FIG. 15A illustrates an ablation assembly.

FIG. 15B illustrates the ablation assembly of FIG. 15A disposed through a Y connector.

FIG. 15C illustrates the ablation assembly of FIG. 15A with catheters disposed therethrough.

FIG. 16A illustrates a handle for an endovenous device.

FIG. 16B illustrates another view of the handle of FIG. 16A.

FIG. 17A illustrates a handle for an endovenous device.

FIG. 17B illustrates the handle of FIG. 17A with an indicator illuminated.

FIG. 18 illustrates a block diagram of an endovenous device.

FIG. 19A illustrates a section view of a handle for an endovenous device.

FIG. 19B illustrates an enlarged section view of a portion of the handle of FIG. 19A.

FIG. 19C illustrates an enlarged section view of a portion of the handle of FIG. 19A.

FIG. 20A illustrates a section view of a handle for an endovenous device.

FIG. 20B illustrates a view of the handle of FIG. 20A with a housing removed.

FIG. 20C illustrates another view the handle of FIG. 20A with the housing removed.

FIG. 20D illustrates a sectioned view of the handle of FIG. 20A.

FIG. 20E illustrates the handle of FIG. 20E with a proximal cap removed.

FIG. 21A illustrates a view of a handle with the housing removed.

FIG. 21B illustrates another view of the handle of FIG. 21A with the housing removed.

FIG. 22 illustrates a table of nonlimiting example voltages (volts), currents (amps), resistances (ohms), times (seconds), powers (watts), and heats (joules) for the endovenous devices described herein.

FIG. 23 illustrates an endovenous device.

FIG. 24 illustrates an enlarged view of an expandable member of the endovenous device of FIG. 23.

FIG. 25 illustrates an enlarged view of a distal portion of the endovenous device of FIG. 23.

FIG. 26 illustrates a section view of a distal portion of the endovenous device of FIG. 23.

FIG. 27 illustrates a section view of a distal portion of the endovenous device of FIG. 23.

FIG. 28 illustrates an enlarged view of an actuator of the endovenous device of FIG. 23.

FIG. 29 illustrates a section view of the actuator of FIG. 28.

FIG. 30A illustrates a slider assembly of the actuator of FIG. 28.

FIG. 30B illustrates a section view of the slider assembly of FIG. 30A.

FIG. 31 illustrates a slide of the slider assembly of FIG. 30A.

FIG. 32A illustrates a lock of the slider assembly of FIG. 30A.

FIG. 32B illustrates the lock of FIG. 32A.

FIG. 33A illustrates an enlarged view of a housing of the actuator of FIG. 28.

FIG. 33B illustrates an enlarged view of the actuator of FIG. 28.

FIG. 33C illustrates a section view of the lock of FIGS. 32A and 32B positioned in the housing of FIG. 33A.

FIG. 33D illustrates a section view of the lock of FIGS. 32A and 32B positioned in the housing of FIG. 33A.

FIG. 33E illustrates a section view of the actuator of FIG. 28.

FIG. 34 illustrates an endovenous device.

FIG. 35A illustrates an enlarged view of the distal portion of the endovenous device of FIG. 34.

FIG. 35B illustrates a side view of the endovenous device of FIG. 34.

FIG. 35C illustrates a section view of the endovenous device of FIG. 34.

FIG. 36 illustrates a section view of the actuator of the endovenous device of FIG. 34.

FIG. 37 illustrates a section view of a distal portion of the endovenous device of FIG. 34.

FIG. 38 illustrates an endovenous device.

FIG. 39A illustrates a side view of the endovenous device of FIG. 38.

FIG. 39B illustrates a side view of a distal portion of the endovenous device of FIG. 38.

FIG. 40 illustrates an expandable member of the endovenous device of FIG. 38.

FIG. 41 illustrates a section view of the expandable member of the endovenous device of FIG. 38.

FIG. 42 illustrates a section view of the endovenous device of FIG. 38.

FIG. 43 illustrates an actuator of the endovenous device of FIG. 38.

FIG. 44 illustrates a section view of the actuator of FIG. 43.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below.

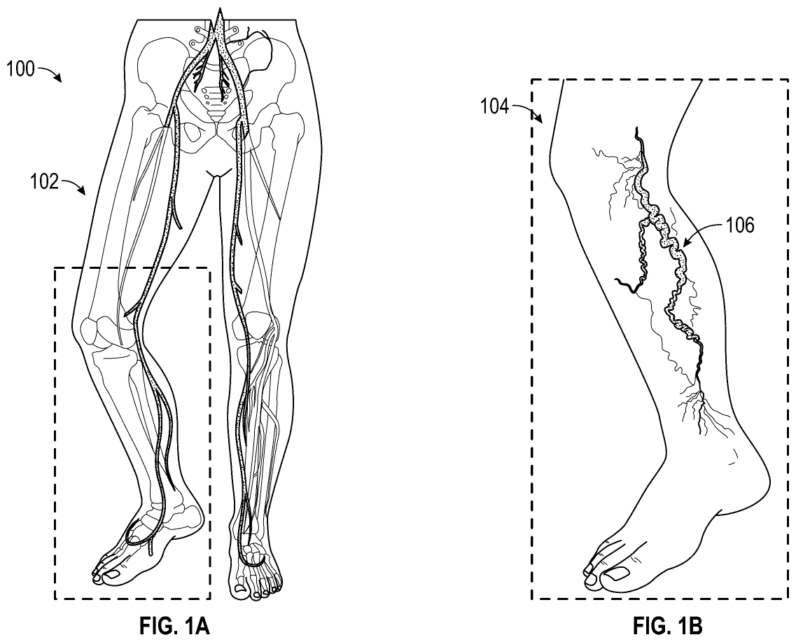

FIG. 1A illustrates a venous system 102 of lower extremities of a patient 100. The venous system 102 includes deep veins and superficial veins that are close to the skin of the patient 100. It is understood that superficial veins can become varicose with increased blood pressure. Increased blood pressure may occur when the valves inside the vein stop working properly, which can be due to a variety of factors such as age, pregnancy, weight, repeated sitting or standing for long periods, an inactive lifestyle, family history, injuries, etc. FIG. 1B illustrates varicose veins 106, which can sometimes be seen on the exterior of the patient as bulging, enlarged, and/or twisted veins. Varicose veins 106, in some instances, are not merely cosmetic but can be accompanied by pain, discomfort, and/or other health problems. Varicose veins 106 can be found in different regions of the human body but are often found to affect the veins in the legs.

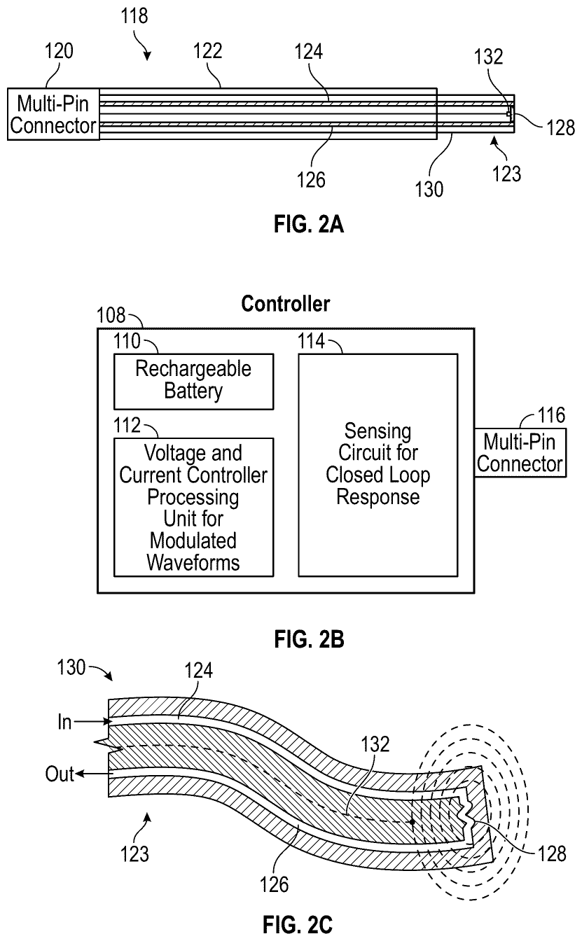

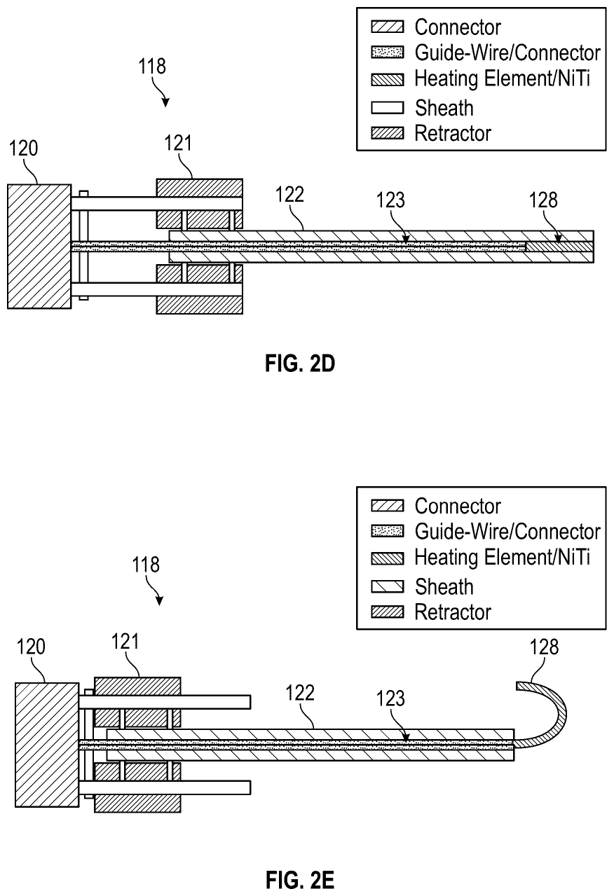

FIG. 2A illustrates an ablation assembly 118, which can be referred to as an ablation catheter and/or ablation catheter assembly, that can be used to treat varicose veins. The ablation assembly 118 can include an elongate element 123 (e.g., catheter, tube, wire, guide wire). The elongate element 123, in some variants, can include an internal lumen through which other devices (e.g., catheter(s), guide wire(s), mechanical agitator(s), etc.) can be disposed. The elongate element 123 can include a heating element 128. The heating element 128 can be disposed at a distal portion (e.g., distal tip) of the elongate element 123. The heating element 128 can convert electrical energy, such as direct current, into heat. The elongate element 123 can be navigated through the vasculature of a patient to position the heating element 128 at the varicose vein. The heating element 128 can heat the inner wall (e.g., endothelium) of the varicose vein to occlude the vein, which can include damaging the vein to occlude with scar tissue and/or causing the vein to collapse to occlude.

The elongate element 123 can include a conductor 124 and/or a conductor 126. The conductor 124 can conduct electrical energy, such as direct current, from a power source (e.g., battery, rechargeable battery, power connection, generator) to the heating element 128. The conductor 126 can conduct electrical energy, such as direct current, away from the heating element 128. The electrical energy flowing through the heating element 128 can heat the heating element 128 with restive heating, which can also be described as Joule heating and/or ohmic heating. The elongate element 123 can include insulation 130 to insulate (e.g., electrically insulate) the conductor 124 and/or conductor 126. For example, the conductor 124 and/or conductor 126 can be coated with the insulation 130 (e.g., enamel coated).

The heating element 128 can have a higher resistance than the conductor 124 and/or conductor 126 to facilitate heating. In some variants, the heating element 128 can have a higher resistance compared to the conductor 124 and/or conductor 126 due to material and/or geometric characteristics. For example, the heating element 128 can include a material with a higher resistance than the conductor 124 and/or conductor 126 such as nickel titanium (e.g., Nitinol). The conductor 124 and/or conductor 126 can include a material with lower resistance (e.g., copper, gold, aluminum, steel, silver, brass, platinum, iron, etc.). The heating element 128 can include a smaller cross-sectional size than the conductor 124 and/or conductor 126 for increased resistance. In some variants, the conductor 124, conductor 126, and/or heating element 128 can include the same or different materials. In some variants, the conductor 124 and/or conductor 126 can include the same or different materials. For example, the conductor 124 to deliver electrical energy to the heating element 128 can include a material with lower resistance, such as copper, to avoid losses while the conductor 126 can include a less expensive material, such as aluminum, to conduct electrical energy away from the heating element 128 when losses may be less important. In some variants, the conductor 124 and/or conductor 126 can include the same material, which can at least include any of the foregoing. In some variants, the heating element 128 can include the same material as the conductor 124 and/or conductor 126 (e.g., the material can be continuous between the conductor 124, conductor 126, and/or heating element 128) but the geometric characteristics (e.g., cross-sectional size) can be varied (e.g., cross-sectional size reduced) to increase resistance to facilitate sufficient resistive heating. In some variants, the heating element 128 can be coupled (e.g., welded, clamped, bonded, clinched, fused) to the conductor 124 and/or conductor 126.

The ablation assembly 118 can include one or more temperature sensors. For example, the elongate element 123 can include a thermocouple 132. The thermocouple 132 can be disposed at and/or proximate the heating element 128 to monitor temperature. The electrical energy flowing through the heating element 128 can be adjusted based on the sensed temperatures at the thermocouple 132. For example, the electrical energy flowing through the heating element 128 can be stopped, reduced, or increased based on the sensed temperature. The wiring for the thermocouple 132 can be routed through the elongate element 123.

The ablation assembly 118 can include a sheath 122, which can be a catheter and/or tube. The sheath 122 can be disposed over the elongate element 123. The heating element 128 can be distally disposed outside of the sheath 122. In some variants, the elongate element 123 can be translated relative to the sheath 122, which can include being retracted within and/or deployed from the sheath 122. The sheath 122 can insulate (e.g., thermally insulate) the vasculature from the elongate element 123. In some variants, a gap (e.g., annular gap) can be disposed between an inner diameter of the sheath 122 and an outer diameter of the elongate element 123 which can be used to deliver one or more substances, such as fluids, agents (e.g., local anesthetic such as lidocaine), sclerosants, glue, therapeutics, medicaments, nutrients, etc. into the vasculature, which avoid subcutaneous injections. For example, the one or more substances can be delivered through a distal opening of the sheath 122 and/or one or more peripheral openings through a side wall of the sheath 122. The one or more substances can be delivered prior to mechanical agitation and/or heat is applied to the varicose vein. In some variants, irrigation and/or aspiration can be performed through the sheath 122. In some variants, a catheter can be deployed, which can include being deployed through the sheath 122 and/or elongate element 123 to deliver the one or more substances and/or facilitate irrigation and/or aspiration. In some variants, the sheath 122 can be advanced distally to cover the heating element 128 and proximally retracted to uncover the heating element 128.

The ablation assembly 118 can include a connector 120, which can be a multi-pin connector. The connector 120 can couple the ablation assembly 118 (e.g., elongate element 123 and/or sheath 122) to a controller system 108 illustrated in FIG. 2B. For example, the conductor 124, conductor 126, and/or thermocouple 132 can be electrically coupled to the controller system 108 by way of the connector 120. As illustrated in FIG. 2B, the controller system 108 can include a connector 116, which can be a multi-pin connector. The connector 116 and the connector 120 can interface to couple the controller system 108 and ablation assembly 118.

The controller system 108 can include features to provide and/or control electrical energy delivered to the heating element 128. For example, the controller system 108 can include a battery 110 (e.g., rechargeable battery, disposable battery), processing unit 112, sensing circuit 114, and/or other hardware. The battery 110 can provide the electrical energy (e.g., direct current) to heat the heating element 128 with resistive heating. For example, direct current can flow from the battery 110 through the conductor 124 to the heating element 128 where the electrical energy is converted into heat. The direct current can flow away from the heating element 128 by way of the conductor 126.

The processing unit 112 can control the voltage and current flowing from the battery 110, which can include stopping, reducing, and/or increasing the voltage and current. The processing unit 112 can modulate waveforms of the electrical current from the battery 110.

The controller system 108 can include a sensing circuit 114 (e.g., thermocouple circuit). The sensing circuit 114 can be connected to the thermocouple 132 to facilitate controlling electrical energy flowing from the battery 110 to the heating element 128. For example, the thermocouple 132 can monitor a temperature at and/or proximate the heating element 128 and the electrical energy flowing from the battery 110 can be controlled (e.g., controlled by the processing unit 112) based on the monitored temperature. The sensing circuit 114 can facilitate closed loop response based on the temperatures sensed by the thermocouple 132. In some variants, the ablation assembly 118 can include a plurality of thermocouples 132 positioned at different locations.

A handle can include the controller system 108. The controller system 108 can be housed within a handle. The controller system 108 and ablation assembly 118 can be coupled together to make a hand-held endovenous device. The handle housing the controller system 108 can be grasped by a clinician to position (e.g., advance, retract, and/or rotate) the ablation assembly 118 (e.g., heating element 128, elongate element 123) within the vasculature. The controller system 108 can include one or more user interfaces, such as buttons, to enable a clinician to control the hand-held endovenous device, which can at least include adjusting the electrical energy delivered to the heating element 128.

As described herein, in some variants, the heating element 128 can include one or more expanding (e.g., self-expanding) features, such as coils, spirals, loops, strips, leads, wires, hooks, etc. The one or more expanding (e.g., self-expanding) features can expand to contact vein walls of different sizes and adjust (e.g., increase, decrease) a size of an outer periphery of the expanding (e.g., self-expanding) features with a changing size of an inner periphery defined by the vein wall. The expanding features can, in some variants, expand when heated by way of electrical energy flowing through the heating element 128. In some variants, the expanding features can self-expand when unsheathed. The expanding features can bring the heating element 128 in contact with the inner wall of the vein to facilitate heat transfer and/or mechanically agitate the inner wall of the vein. In some variants, the heating element 128 can mechanically agitate the inner wall of the vein prior to the application of heat or heat can be applied prior to mechanical agitation. In some variants, the ablation assembly 118 and/or another device for use with the ablation assembly 118 can include expanding (e.g., self-expanding) features separate from the heating element 128. In some variants, the separate expanding (e.g., self-expanding) features can mechanically agitate the inner wall of the vein prior to, simultaneously with, and/or after the application of heat by the heating element 128, which can, in some variants, enable a lower temperature to be used.

In some variants, the processing unit 112 can switch the direction of the current so that the current flows to the heating element 128 through the conductor 126 and flows away from the heating element 128 through the conductor 124. In some variants, the processing unit 112 can quickly switch flow for bi-directional to provide a variable frequency. The variable frequency can at least be less than 3 kHz, greater than 300 GHz, or between 3 kHz and 300 GHz.

FIG. 2C illustrates the heating element 128 being heated with resistive heating by direct current. Direct current can flow through the conductor 124 to the heating element 128 and flow away from the heating element 128 by way of the conductor 126. As described herein, the heating element 128 can include expanding features, such as self-expanding features, to put the heating element 128 in contact with the inner wall of the vein.

As illustrated in FIGS. 2D and 2E, the ablation assembly 118 can include an actuator 121 (e.g., retractor) that can move the elongate element 123 relative to the sheath 122 and/or sheath 122 relative to the elongate element 123 to unsheathe the heating element 128. The actuator 121 can be manually operated, include one or more springs biasing the elongate element 123 and/or sheath 122 toward a configuration, and/or include automated features. When unsheathed, the heating element 128 can self-expand to contact the inner wall of the vein. In some variants, the heating element 128 can self-expand when heated by electrical energy.

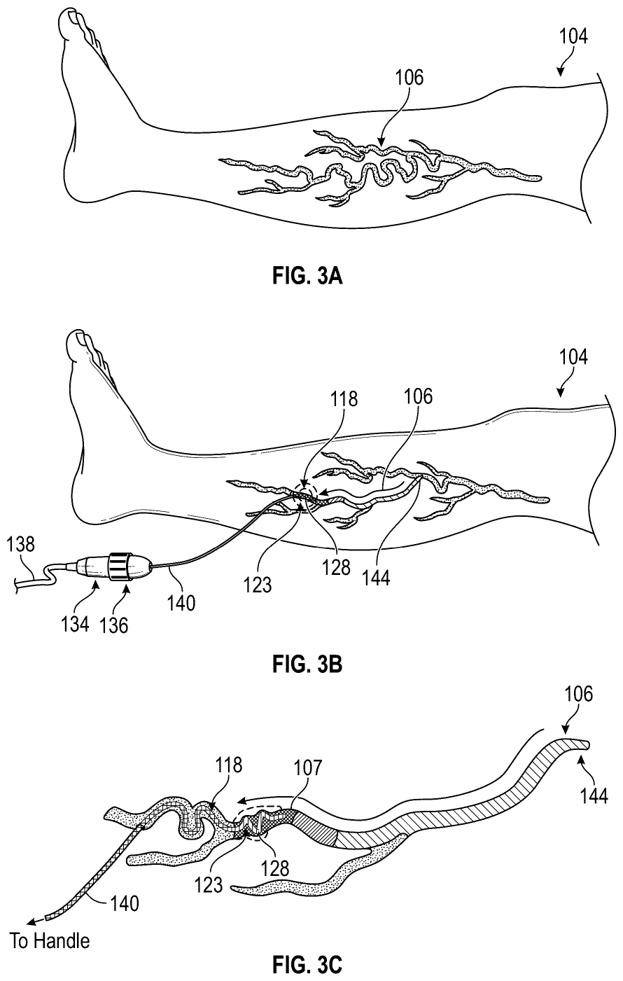

FIG. 3A illustrates a leg 104 with varicose veins 106. As illustrated in FIGS. 3B and 3C, an endovenous device 134 can be used to treat varicose veins 106. Vascular access can be obtained using percutaneous techniques. An elongate element 123 of an ablation assembly 118 of the endovenous device 134 can be navigated through the vasculature to position the heating element 128, which can include a coil shape, within a varicose vein 106. A sheath 140 can be disposed over a portion (e.g., proximal portion) of the elongate element 123 to thermally insulate the vasculature from the elongate element 123. In some variants, the ablation assembly 118 (e.g., sheath 140 and/or elongate element 123) can be advanced over a guide wire to position the heating element 128 within a varicose vein 106. The endovenous device 134 can include a handle 136 to facilitate manipulating (e.g., retracting, advancing, rotating, steering) the ablation assembly 118 within the vasculature. The handle 136 can house a controller to control electrical energy delivered to the heating element 128, which can include reducing, increasing, or stopping the delivery of electrical energy. The handle 136 can include a battery and/or a power cable 138 connected to a power source to provide electrical energy to heat the heating element 128 with resistive heating.

In use, the ablation assembly 118 can be advanced through a varicose vein 106 to position the heating element 128 at a distal position 144 within a varicose vein 106. In some variants, a guide wire can be navigated to within a varicose vein, the sheath 140 (e.g., delivery sheath) can be distally advanced over the guide wire to the varicose vein, and the elongate element 123 can be distally advanced through the lumen of the sheath 140 to the varicose vein. In some variants, the guide wire can be retracted prior to insertion of the elongate element 123. In some variants, the elongate element 123 and/or sheath 140 can be positioned at the varicose vein without a guide wire. In some variants, the elongate element 123 can be distally advanced over the guide wire. In some variants, the sheath 140 and elongate element 123 can be distally advanced together to the varicose vein.

Electrical energy can flow through the heating element 128 to raise a temperature of the heating element 128 to heat the vein wall 107. As shown in FIG. 3C, the heating element 128 can include a coil (e.g., self-expanding coil). In some variants, the self-expanding coil can self-expand when electrical energy has flowed through the heating element 128 to raise the temperature of the heating element 128 to a threshold. In some variants, the self-expanding coil can self-expand when unsheathed by the sheath 140. The coil can expand (e.g., self-expand) to contact the vein wall 107, which can include self-expanding from a different shaped configuration such as a straight wire to a coil. The coil contacting the vein wall 107 can mechanically agitate the vein wall 107 and/or facilitate improved heat transfer from the coil to the vein wall 107 to damage the vein wall 107. The mechanical agitation and/or heat from the heating element 128 can cause the varicose vein 106 to collapse, which can occlude the varicose vein 106. The ablation assembly 118 (e.g., elongate element 123 with the heating element 128) can be proximately retracted to mechanically agitate and/or heat a length of the vein wall 107 with the heating element 128 to collapse a length of the varicose vein 106. The outer diameter of the coil of the heating element 128 can automatically change (e.g., increase, decrease) to an inner diameter defined by the vein wall 107 to maintain contact between the coil of the heating element 128 and the vein wall 107 as the heating element 128 is proximally retracted. As described herein, the endovenous device 134 and/or another device can be used to introduce one or more substances, such as an agent (e.g., numbing agent), into the vasculature and/or facilitate irrigation and/or aspiration. After treatment, the ablation assembly 118 can be proximally retracted out of the leg 104.

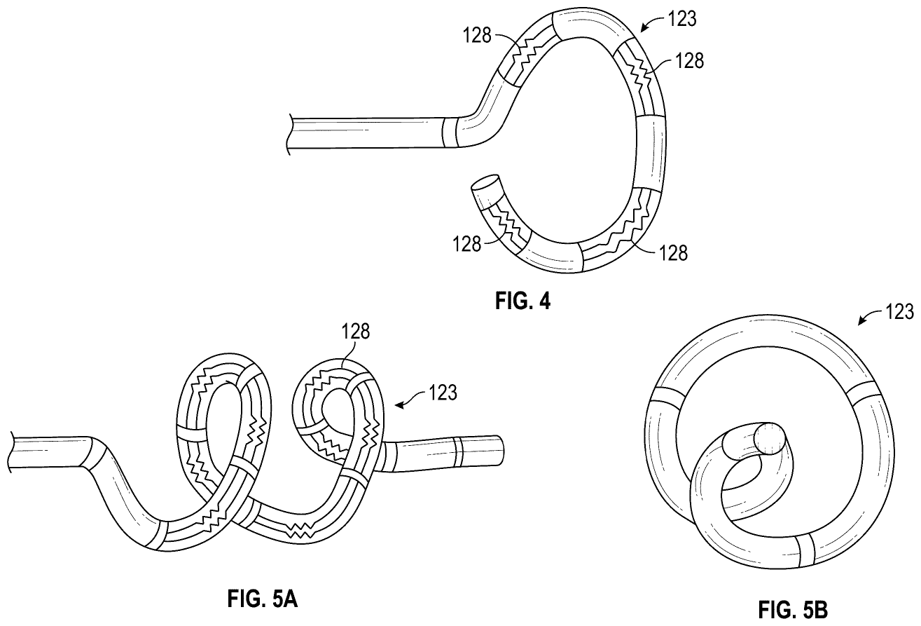

As described herein, the elongate element 123 can include one or more self-expanding features, which can include a self-expanding heating element 128. As illustrated in FIG. 4, the elongate element 123 can include a self-expanding loop, which can be disposed at a distal portion of the elongate element 123. The self-expanding loop can include one or more heating elements 128 (e.g., a plurality of heating elements 128). The heating element 128 can be distributed along the self-expanding loop. The elongate element 123 can include one or more markers to facilitate tracking in the body of the patient. The self-expanding loop can change an outer diameter thereof to follow a changing inner diameter of the vein during proximal retraction.

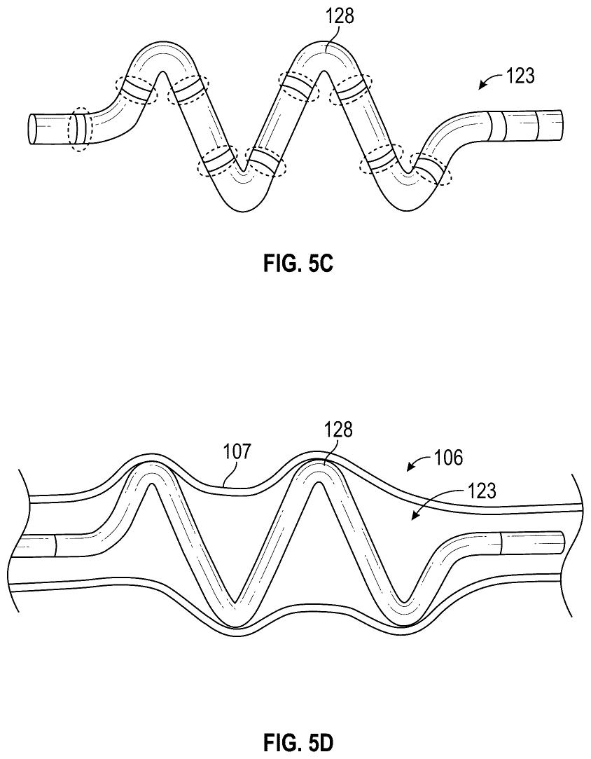

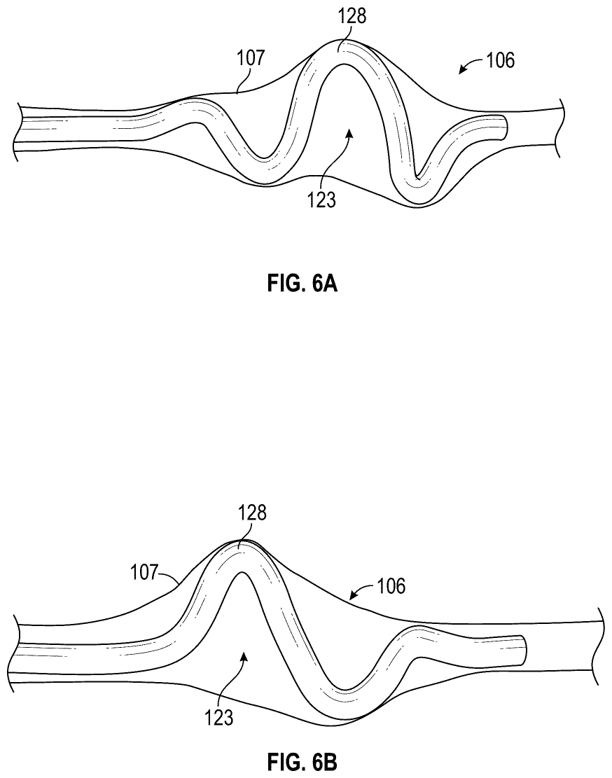

FIGS. 5A-5D illustrate an elongate element 123 with a heating element 128 that includes a self-expanding coil. The heating element 128 can include one or more markers to facilitate tracking in the body of the patient. The heating element 128 can include one, two, three, four, five, six, or more coils. As illustrated in FIG. 5D, the coils of the heating element 128 can self-expand to contact the vein wall 107. As the elongate element 123 is retracted, the coils of the heating element 128 can maintain contact with the vein wall 107 to mechanically agitate the vein wall 107. The contact between the coils of the heating element 128 and the vein wall 107 can facilitate heat transfer to the vein wall 107 to damage the vein wall 107. FIG. 6A illustrates that the coils of the heating element 128 can include a tapered outer periphery, which can include being tapered in a distal-proximal direction and/or proximal-distal direction. In some variants, the outer periphery of the coils can increase in size in a distal direction to a midportion and then decrease.

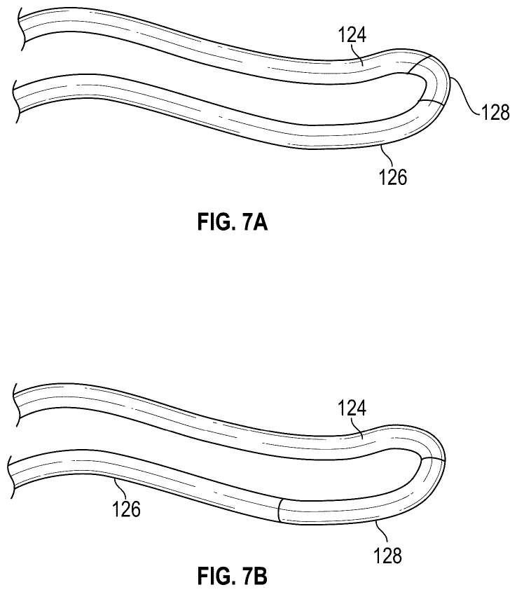

FIGS. 7A and 7B illustrate the conductor 124, conductor 126, and heating element 128. As shown, the conductor 124, conductor 126, and heating element 128 can form a loop that extends distally and then proximally. The heating element 128 can include varying lengths, which can least include 1-10 centimeters. In some variants, the length of the heating element 128 can be less than 1 centimeter. The heating element 128 can be a point in some variants. In some variants, the heating element 128 can be more than 10 centimeters. In some variants, the heating element 128 can be a continuation of the conductor 124 and/or conductor 126 but with a geometry (e.g., cross-section) for increased resistance. In some variants, the heating element 128 can be coupled (e.g., welded, clamped, bonded, clinched, fused) to the conductor 124 and/or conductor 126.

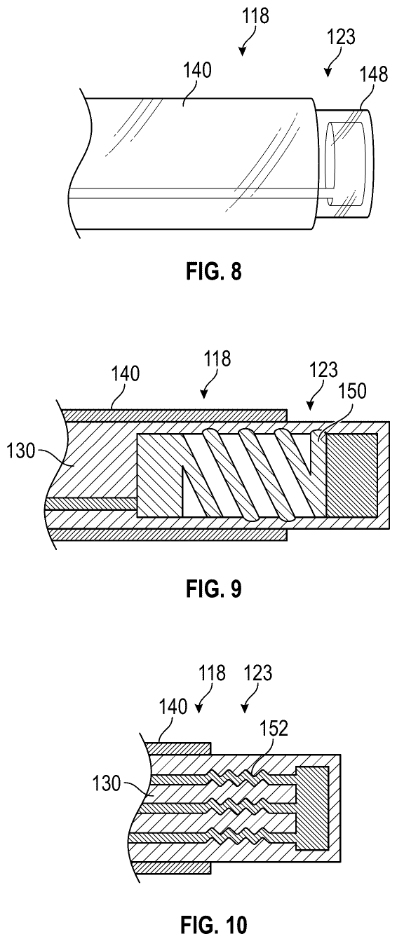

The heated element can include various configurations. As illustrated in FIG. 8, the elongate element 123 can include a heated element ring 148. As illustrated in FIG. 9, the elongate element 123 can include a heated element coil 15. As illustrated in FIG. 10, the elongate element 123 can include a plurality of heated element strips 152 (e.g., leads), which can be circumferentially distributed around a distal portion of the elongate element 123.

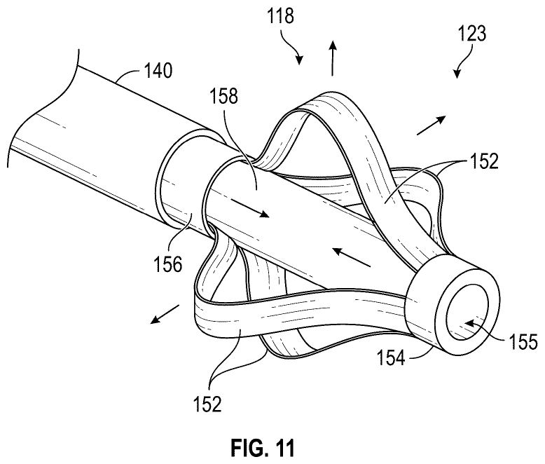

The heated element can include expanding features that expand in response to mechanical manipulation. As illustrated in FIG. 11, the elongate element 123 can include a plurality of heated element strips 152. The elongate element 123 can include an outer tube 156 disposed over an inner tube 158. The inner tube 158 can be translated relative to the outer tube 156 which can include being proximally retracted into and distally disposed out of the outer tube 156. The heated element strips 152 can be coupled to a distal portion of the outer tube 156 and a distal portion of the inner tube 158, which can include being coupled to a distal tip 154 (e.g., distal ring) of the inner tube 158. The inner tube 158 can be distally advanced relative to the outer tube 156 to collapse the heated element strips 152, which can include moving the heated element strips 152 toward the inner tube 158. The inner tube 158 can be proximally retracted relative to the outer tube 156 to expand the heated element strips 152, which can include moving a middle portion of the heated element strips 152 away from the inner tube 158 as shown in FIG. 11. The heated element strips 152 can be expanded (e.g., moved away from the inner tube 158) to contact the inner wall of the vein.

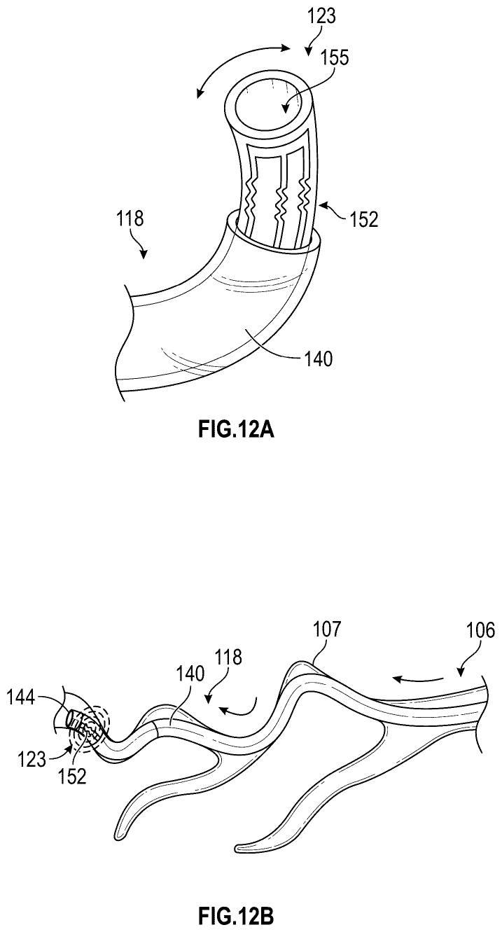

FIG. 12A illustrates the elongate element 123 with the plurality of heated element strips 152 disposed on a distal portion of the elongate element 123 that is distally disposed out of the sheath 140. The elongate element 123 can be steerable to help navigate through the vasculature. For example, as illustrated in FIG. 12B, the elongate element 123 and sheath 140 can be navigated through the tortuous path of a varicose vein 106. The heated element strips 152 can be expanded as described herein to contact the vein wall 107 which can mechanically abrade the vein wall 107 and/or heat. In some variants, as illustrated in FIG. 12A, the elongate element 123 can include a lumen 155 through which a guide wire and/or one or more catheters and/or other devices can be disposed.

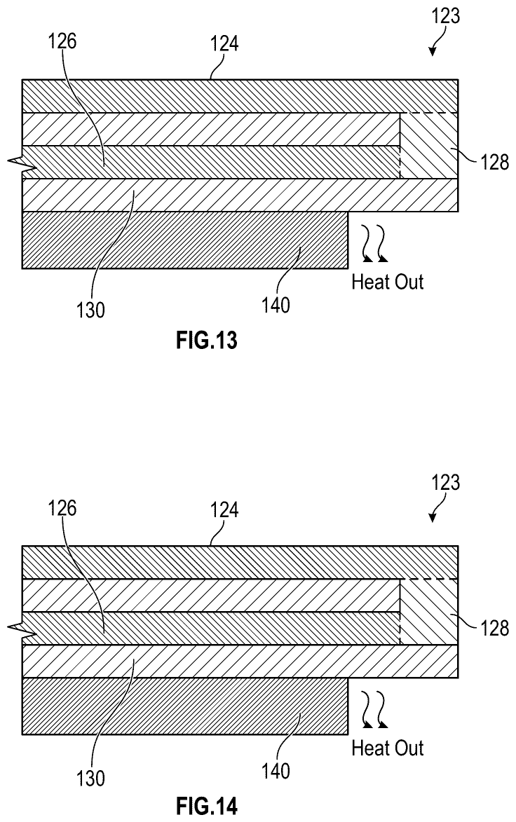

FIG. 13 illustrates an elongate element 123 with a sheath 140. As shown, the elongate element 123 includes a conductor 124, conductor 126, and heating element 128. The conductor 124 and conductor 126 can include the same material and/or cross-sectional shape. The heating element 128 can include a different material and/or cross-sectional shape compared to the conductor 124 and conductor 126. The heating element 128 can include various configurations such as a coil or ring. The heating element 128 can be coupled (e.g., welded) to the conductor 124 and conductor 126. As shown in FIG. 14, the conductor 124 and conductor 126 can include different materials and/or cross-sectional shapes relative to each other.

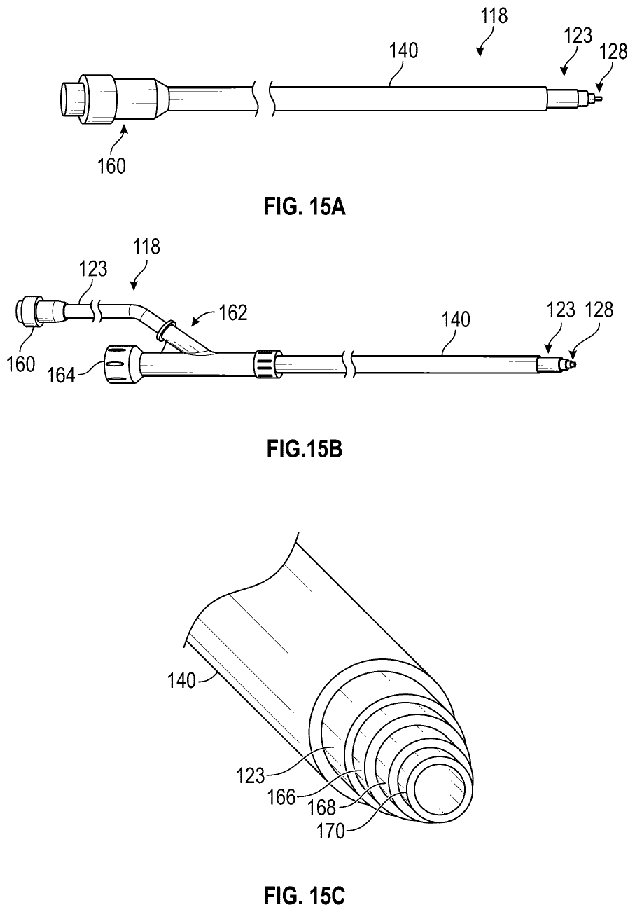

FIG. 15A illustrates an ablation assembly 118 with an elongate element 123, sheath 140, and connector 160. The connector 160, as described herein, can connect the elongate element 123 to a controller to deliver electrical energy (e.g., direct current) to the heating element 128 of the elongate element 123. The sheath 140 can be disposed over the elongate element 123 to thermally insulate the vasculature from the elongate element 123. The heating element 128 that can be distally disposed outside of the sheath 140. The sheath 140 can be coupled to the connector 160. As illustrated in FIG. 15B, the sheath 140, which can be a catheter, can be disposed on a distal end of a Y connector 162 (e.g., Y adapter). The elongate element 123 can be disposed into the Y connector 162 (e.g., through the angled port of the Y connector 162) and through the sheath 140 to position the heating element 128 distally out of the sheath 140. The Y connector 162 can include a valve 164 (e.g., Tuohy Borst adapter, hemostasis valve) coupled thereto (e.g., coupled to the straight proximal port of the Y connector 162). Other devices can be routed through one or more of the valve 164, Y connector 162, sheath 140, and/or elongate element 123. For example, a guide wire and/or catheter can be disposed through the valve 164, Y connector 162, and/or sheath 140. As illustrated, in some variants, the ablation assembly 118 can be used in conjunction with one or more catheters. In some variants, the elongate element 123 can be disposed in the sheath 140. In some variants, one or more catheters can be disposed through the sheath 140. In some variants, one or more catheters can be disposed through the elongate element 123. For example, a catheter 166 can be disposed through the elongate element 123, a catheter 168 can be disposed through the catheter 166, and/or a catheter 170 can be disposed through the catheter 168 as shown in FIG. 15C. Various catheters can be used for various purposes such as irrigation, aspiration, delivery of one or more substances, delivery of one or more devices, a guide wire, etc.

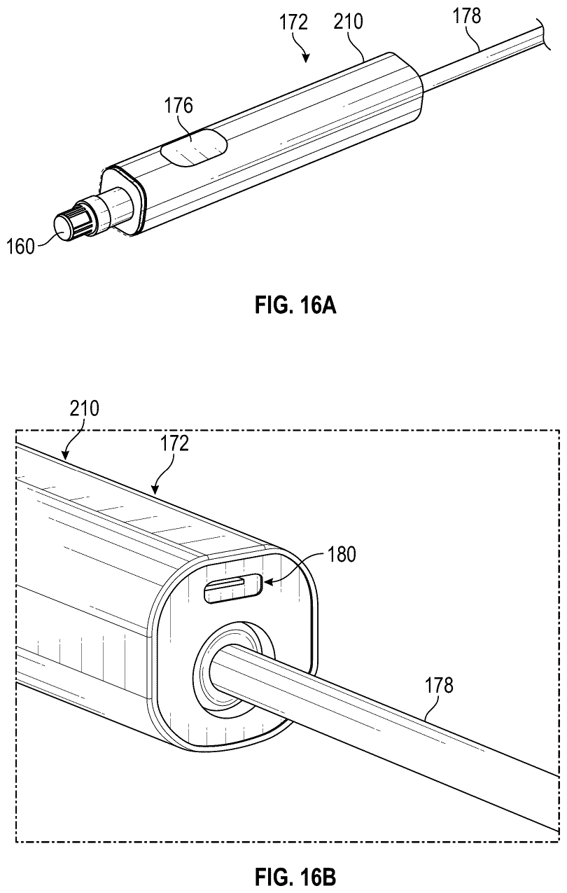

FIG. 16A illustrates a handle 172 for an endovenous device. The handle 172 can include a housing 210 to house a controller system, which can include a battery to power the endovenous device. In some variants, the handle 172 can be connected to an external power source with a cable 178. An elongate element 123 (not shown in FIG. 16A) and/or ablation assembly can be coupled to the handle 172 with a connector 160. The connector 160 can couple to a connector of the handle 172. The handle 172 can include a user interface 176 (e.g., button, switch, toggle, slide, dial) to enable a clinician to control the delivery of electrical energy by the control system to the heated element of the endovenous device. For example, the clinician can press the user interface 176 to deliver electrical energy to the heated element to raise a temperature of the heated element and release the user interface 176 to cease delivering electrical energy. As shown in FIG. 16B, the handle 172 can include a port 180 (e.g., USB-C) to charge a battery in the handle 172. As described herein, the handle 172 can be manipulated by a clinician to maneuver (e.g., advance, retract, rotate) the elongate element 123 and/or sheath 140 within the vasculature of a patient.

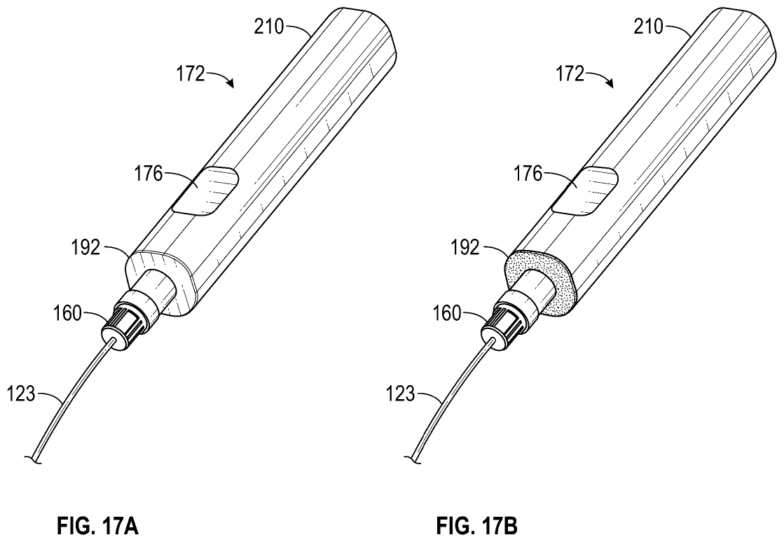

FIG. 17A illustrates the handle 172 with an elongate element 123 connected to the connector 160. The control system disposed within the handle 172 can deliver electrical energy (e.g., direct current) to a heated element of the elongate element 123. The handle 172 can include an indicator light to visually indicate to a clinician that the control system is delivering electrical energy to the heated element. For example, the handle 172 can include a lens 192 that can be illuminated, as illustrated in FIG. 17B, when the control system is delivering electrical energy to the heated element.

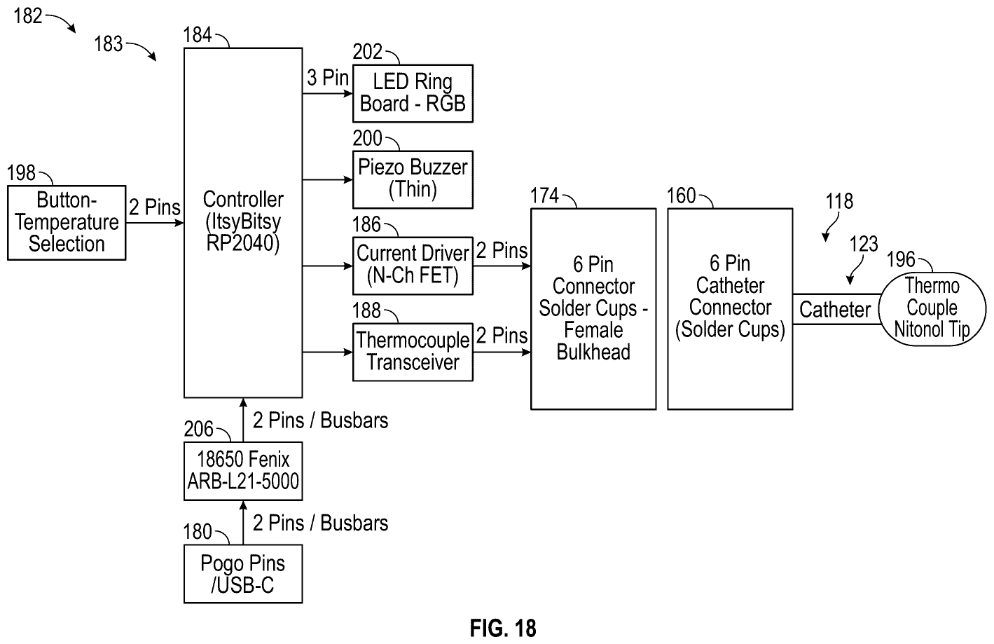

FIG. 18 illustrates a block diagram for an endovenous device 182. The endovenous device 182 can include a control system 183 and an ablation assembly 118. The control system 183 can power and/or control the ablation assembly 118. The control system 183 can be housed within a handle. The handle can be used to move (e.g., rotate, advance, retract, steer, etc.) the ablation assembly 118 through the vasculature.

The control system 183 can include a controller 184, button assembly 198, visual indicator circuit board 202, buzzer 200, current driver 186, thermocouple transceiver 188, battery 206, port 180, connector 174, and/or other components. The controller 184 can implement the various functions to control the endovenous device 182 described herein. The button assembly 198 can enable a user to interact with (e.g., push) a button to control the endovenous device 182, which can include controlling the delivery of electrical energy (e.g., direct current) to the heated element of the ablation assembly 118. The battery 206, which can be rechargeable and/or disposable, can power the endovenous device 182. The control system 183 can include a port 180 (e.g., USB-C port) that can interface with a power connector to charge the battery 206.

The control system 183 can include a visual indicator circuit board 202 (e.g., LED ring board) that is powered by the battery 206. The visual indicator circuit board 202 can illuminate a feature, such as a lens (e.g., ring lens) of the handle. The visual indicator circuit board 202 can illuminate a feature when the battery 206 is delivering electrical energy (e.g., direct current) to heat the heated element of the ablation assembly 118. The visual indicator circuit board 202 can illuminate a feature, which can include blinking, when the charge of the battery 206 decreases to a threshold. In some variants, the visual indicator circuit board 202 can emit different colors of light, intensities, and/or patterns (e.g., blinking patterns) to visually communicate warnings and/or information.

The control system 183 can include a buzzer 200 (e.g., piezo buzzer) that is powered by the battery 206. The buzzer 200 can emit a sound. The buzzer 200 can emit a sound when the temperature sensor (e.g., thermocouple) of the control system 183 senses a threshold temperature. In some variants, the buzzer 200 can emit a sound when the battery 206 is delivering electrical energy (e.g., direct current) to heat the heated element of the ablation assembly 118. In some variants, the buzzer 200 can emit a sound when the charge of the battery 206 decreases to a threshold. In some variants, the buzzer 200 can emit different sounds, intensities, and/or patterns to audibly communicate warnings and/or information.

The control system 183 can include a current driver 186 to facilitate delivering electrical energy from the battery 206 to the heated element and/or thermocouple of the ablation assembly 118. The control system 183 can include a thermocouple transceiver 188 to facilitate receiving data from the thermocouple of the ablation assembly 118 to monitor temperature.

The control system 183 can include a connector 174 (e.g., multi-pin connector, six-pin connector, female connector). The connector 174 can couple (e.g., interface) with a connector 160 (e.g., multi-pin connector, six-pin connector, male connector) of the ablation assembly 118. The coupling between the connector 174 and connector 160 can facilitate the transfer of electrical energy, data, and/or instructions between the control system 183 and ablation assembly 118. The coupling between the connector 174 and the connector 160 can enable a handle housing the control system 183 to be maneuvered to maneuver the ablation assembly 118, which can include maneuvering within the vasculature.

The endovenous device 182 can include an ablation assembly 118. The ablation assembly 118 can include the connector 160 to facilitate coupling the control system 183 with the ablation assembly 118. The ablation assembly 118 can include the elongate element 123, which can be a catheter. The ablation assembly 118 can include a sheath that can be disposed over a portion of the elongate element 123 which can, in some variants, thermally insulate the vasculature from the elongate element 123. The elongate element 123 can include at least two conductors. One of the two conductors can direct electrical current to the heated element and the other of the two conductors can direct electrical current away from the heated element. In some variants, the endovenous device 182 can alternate the direction that the current flows (e.g., the role of the two conductors can be switched). The elongate element 123 can include insulation to electrically insulate the two conductors.

The elongate element 123 can include a distal portion 196 (e.g., distal tip). The distal portion 196 can include a thermocouple. The distal portion 196 can include a heated element. The heated element can be coupled, which can include using any of the methods described herein, to the conductors of the elongate element 123. Electrical energy (e.g., direct current) can be delivered from the battery 206 to the heated element by way of one of the two conductors of the elongate element 123 and directed away from the heated element by the other of the two conductors of the elongate element 123. As described herein, the elongate element 123 can include expanding features, such as self-expanding features, to contact the inner wall of a vein, which can agitate the inner wall of the vein to occlude the vein. In some variants, the heated element can include one or more expanding features, such as self-expanding features, to contact the heated element with the inner wall of the vein. The one or more self-expanding features can automatically adjust an outer periphery of the one or more self-expanding features to a size of an inner diameter of a vein to maintain contact. The heated element, which can include the self-expanding features, can include a shape memory material such as nickel titanium. The self-expanding features, when not part of the heated element, can include a shape memory material such as nickel titanium.

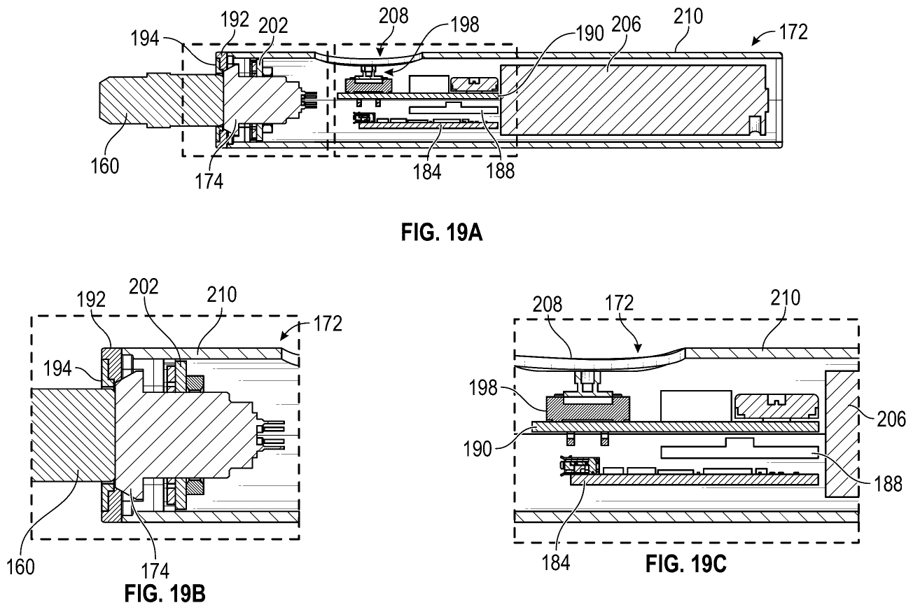

FIGS. 19A-19C illustrate views of a sectioned handle 172 of an endovenous device to show the internal components thereof, which can include components of the control system. As illustrated, the handle 172 can include a housing 210 to house the components of the control system. The handle 172 can include a connector 174 (e.g., multi-pin connector). The connector 174 can be disposed inside the handle 172 but be accessible to the connector 160 of the ablation assembly 118. The connector 174 can be disposed in a distal portion of the handle 172.

The handle 172 can house a visual indicator circuit board 202 (e.g., LED ring board, RGB LED ring board). The visual indicator circuit board 202 can cause light to be emitted through a lens 192 of the handle 172, which can include even intensity. The lens 192 can include a translucent material. The lens 192 can include an annular shape (e.g., ring shape), which can extend around the connector 174. The lens 192 can be molded to facilitate omnidirectional indication.

The handle 172 can house a distal cap 194. The distal cap 194 can hide the bulkhead. The distal cap 194 can enable the positioning of the connector subassembly shown in FIG. 19B to be driven by the lens 192. The subassembly shown in FIG. 19C can be assembled first giving priority to the button assembly and housing 210 relation.

The handle 172 can house an opening 208 to receive a button (e.g., snap on button). The opening 208 can extend through a peripheral wall of the housing 210. The button can interact with a button assembly 198 to receive user commands. For example, in some variants, the button can be pushed to deliver electrical energy (e.g., direct current) to a heated element for heating. In some variants, the button can be held to deliver electrical energy and cease delivering when released.

The handle 172 can house a board 190. The board 190 can be a field effect transistor, buzzer (e.g., piezo buzzer), and/or button board, which can include electronic components to operatively support the foregoing features. The handle 172 can house a thermocouple transceiver 188 (e.g., thermocouple reader) to receive data from and/or send data to a thermocouple of the elongate element 123. The handle 172 can house a controller 184 to carry out the functions described herein. The handle 172 can house a battery 206, which can be rechargeable or single use. The battery 206 can provide power to the electrical components described herein.

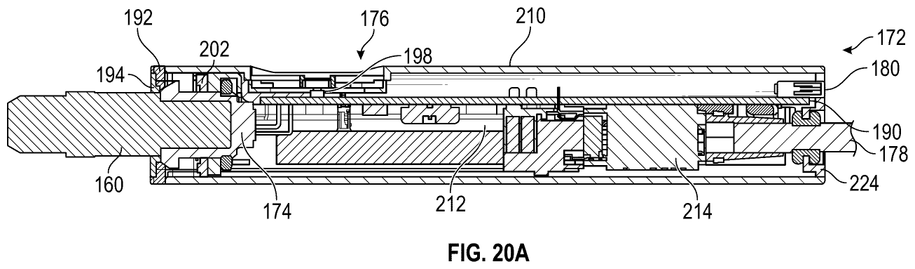

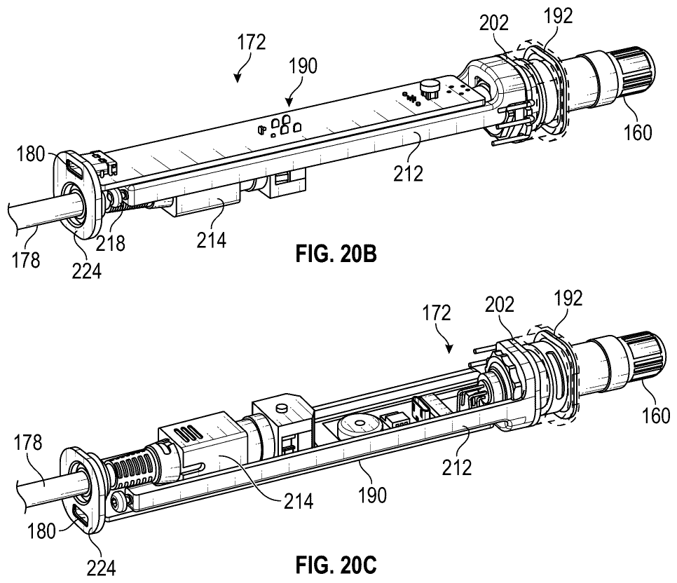

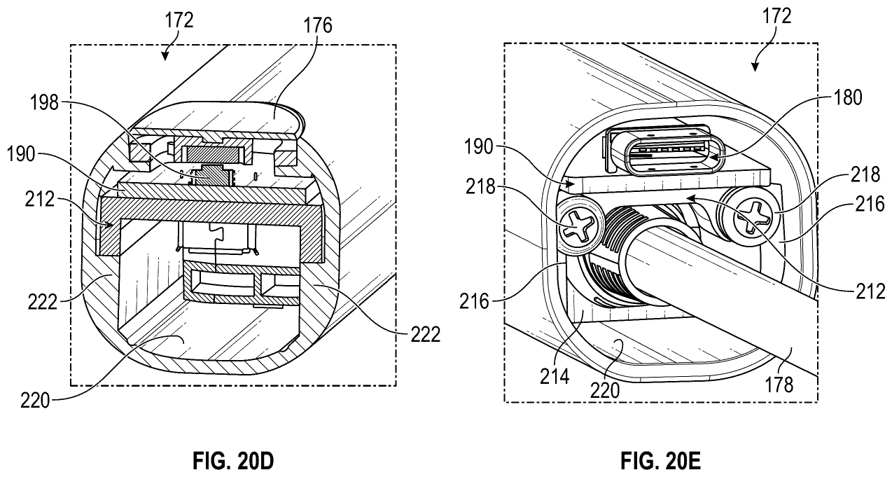

FIG. 20A illustrates a cross section of the handle 172 with a chassis 212. The components housed within the handle 172 can be coupled to the chassis 212 prior to insertion into the housing 210 as shown in FIGS. 20B and 20C, which can facilitate efficient assembly. For example, the connector 174, visual indicator circuit board 202, button assembly 198, board 190, port 180 (e.g., USB-C port), and/or other features can be coupled to the chassis 212 prior to insertion into the housing 210. A battery can be coupled to the chassis 212, which can include prior to insertion into the housing 210. A power connector assembly 214, which can facilitate connecting to a power cable 178 interfaced with a power source, can be coupled to the chassis 212. The power connector assembly 214 and cable 178 can facilitate powering the endovenous device with an external power source such as an outlet, generator, and/or external battery.

The chassis 212 with the internal components coupled thereto can be inserted into an interior 220 (e.g., slot) of the housing 210 as illustrated in FIG. 20D. The housing 210 can include edges 222 (e.g., rails, shelves) upon which the chassis 212 can slide to position the chassis 212 and components coupled thereto within the housing 210. The edges 222 can help correctly position the chassis 212 and internal components within the housing. The chassis 212 and components coupled thereto can be advanced into the interior 220 through a distal opening of the housing 210. The chassis 212 can be advanced through the distal opening until contacting flanges 216 of the housing 210 as shown in FIG. 20E which can correctly position the chassis 212 and internal components coupled thereto. The flanges 216 can be disposed at a proximal portion of the housing 210 and impede further advancement of the chassis 212. The chassis 212 can be secured to the flanges 216 with one or more fasteners 218 (e.g., screws), which can fix the chassis 212 relative to the housing 210. A proximal cap 224 can be disposed over a proximal opening of the housing 210 with the chassis 212 secured within the housing 210. The lens 192 and/or distal cap 194 can be disposed over a distal opening of the housing 210 the chassis 212 secured within the housing 210.

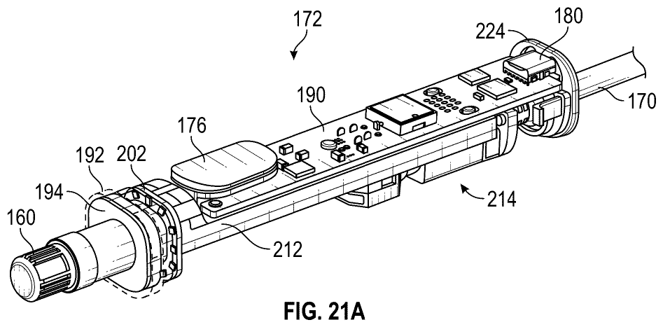

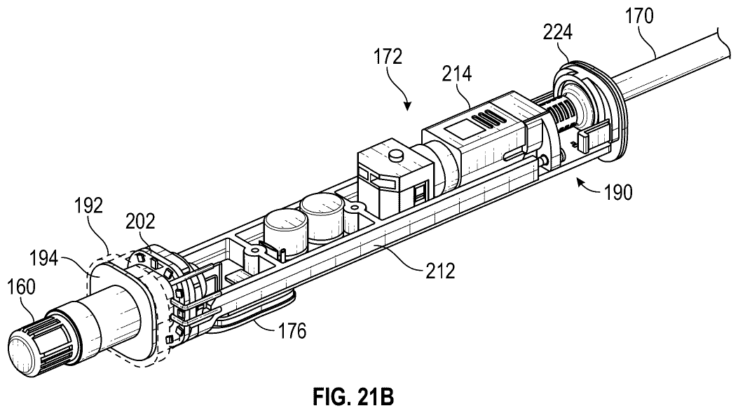

FIGS. 21A and 21B illustrate various views of the handle 172 with a housing removed.

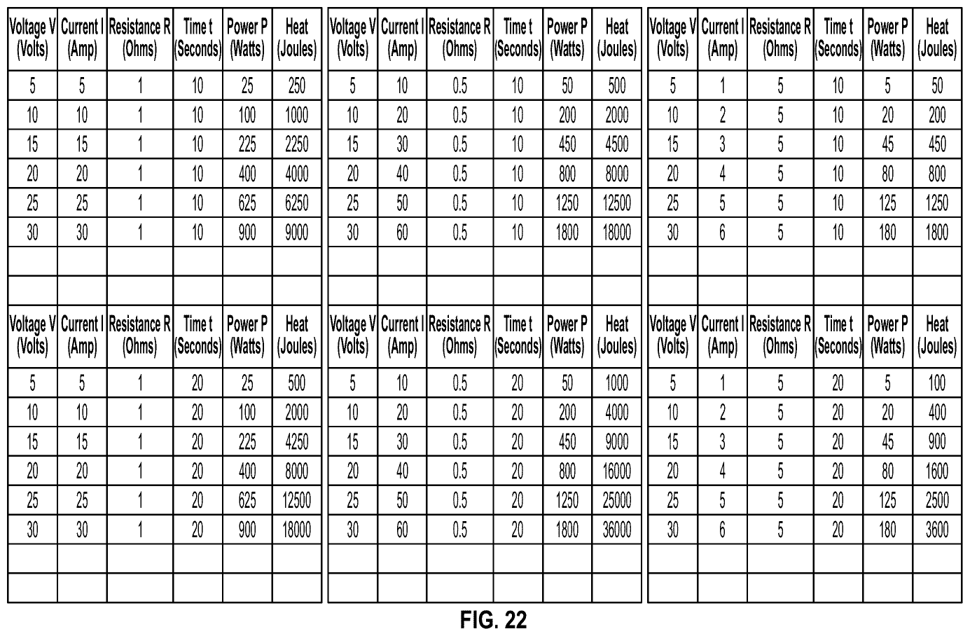

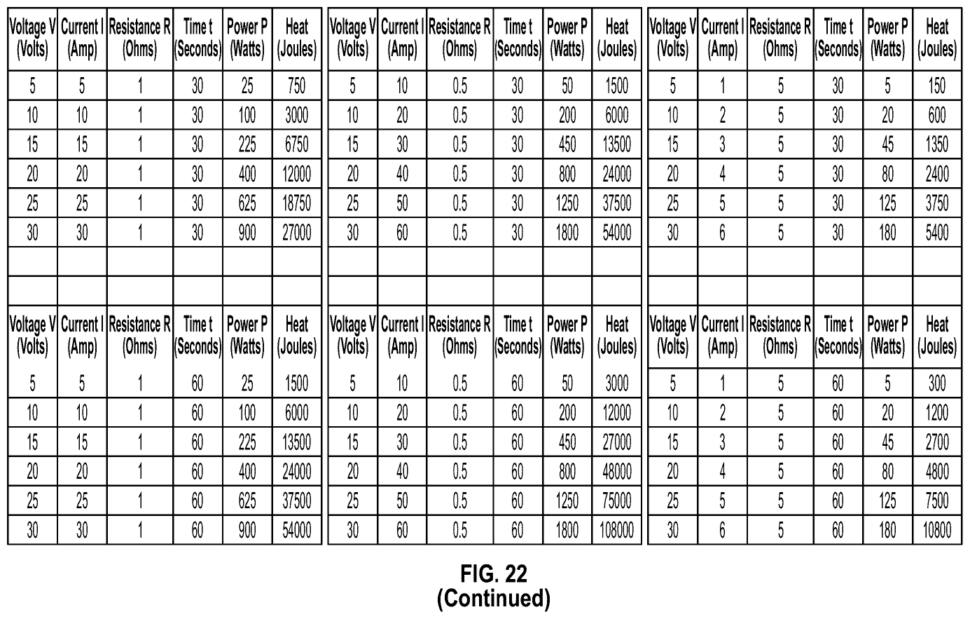

FIG. 22 illustrates a table of nonlimiting example voltages (volts), currents (amps), resistances (ohms), times (seconds), powers (watts), and heats (joules) for the endovenous devices described herein, which can include example parameters for heating the heated elements (e.g., expandable members) described herein with resistive heating by direct current. The endovenous devices described herein can employ any of the values for the parameters shown in FIG. 22, including any value between the listed values and/or range of values bound by two of the listed values or two values between the listed values. In some variants, the power sources (e.g., batteries) described herein can include different voltages, which can at least include 0-50 volts or 0-15 volts. In some variants, the power sources (e.g., batteries) described herein can include different powers, which can at least include 0-2000 watts or 0-100 watts. When current with frequency is employed, the frequency can at least be 0-300 GHz. The current can include 0-30 amperes or 5-20 amperes. The resistance of the heating elements described herein can vary, which can at least include 0.01-1000 ohms, 0.1 to 100 ohms, or 0.5-10 ohms. Current can be applied to the heating elements described herein for various periods of time which can at least include 0-500 seconds or 0-60 seconds.

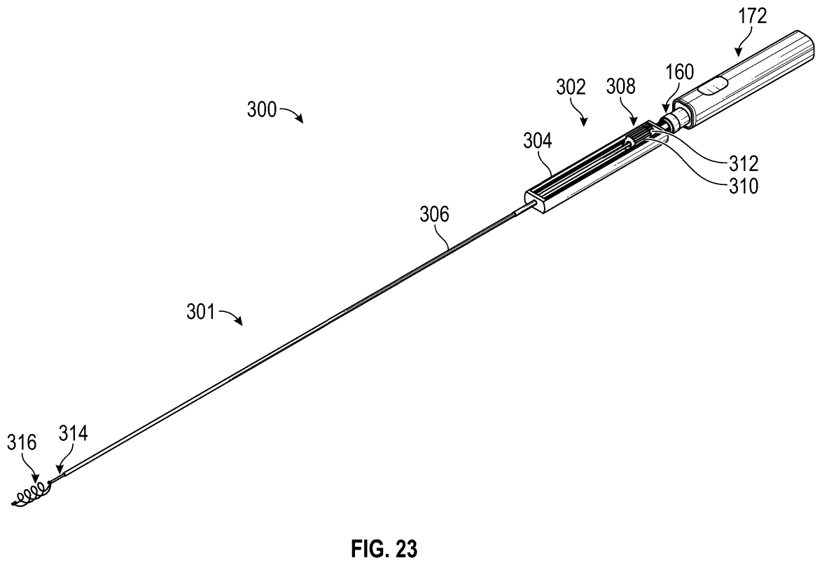

FIG. 23 illustrates an endovenous device 300, which can also be referred to as an ablation device. The endovenous device 300 can be used to treat varicose veins. The endovenous device 300 can include an ablation assembly 301, which can also be referred to as an ablation catheter and/or ablation catheter assembly. The ablation assembly 301 can be coupled to the handle 172. For example, the ablation assembly 301 can be coupled to the handle 172 by way of the connector 160. The handle 172 can provide and control energy (e.g., electrical, direct current, alternating current, radio frequency, non-radio frequency) to the ablation assembly 301. The handle 172 can be used to control the ablation assembly 301.

The ablation assembly 301 can include an elongate element 314, which can also be referred to as an elongate member, catheter, and/or inner catheter. The elongate element 314 can include an expandable member 316 (e.g., self-expanding member, heated element). The expandable member 316 can be disposed at a distal portion of the elongate element 316.

The ablation assembly 301 can include a tube 306 (e.g., outer tube, sheath) that can be disposed over the elongate element 314. In some variants, the tube 306 can be advanced over or retracted to uncover the elongate element 314. In some variants, the tube 306 can be advanced to cover or retracted to uncover the expandable member 316. In some variants, the elongate element 314 can be advanced distally out of the tube 306 or retracted proximally into the tube 306.

The ablation assembly 301 can include an actuator 302 (e.g., actuation mechanism, actuation device) that can be used to cover and uncover the expandable member 316. The actuator 302 can be used to advance the tube 306 over the elongate element 314 (e.g., expandable member 316) and/or retract the tube 306 to uncover the elongate element 314 (e.g., expandable member 316). In some variants, the actuator 302 can be used to advance the elongate element 314 relative to the tube 306 to cover and uncover the expandable member 316.

The actuator 302 can include a housing 304. The tube 306 and/or another feature (e.g., tube) coupled to the tube 306 can extend through the housing 304 to couple with a slider assembly 308. The slider assembly 308 can slide within the housing 304, which can include axial advancement and/or retraction within the housing 304, to move the tube 306 relative to the elongate element 314. The slider assembly 308 can include a slide 310 and/or lock 312. The slide 310 and the lock 312 can be coupled together, which can include being rotatably coupled together. The tube 306 can be coupled to the slide 310. The lock 312 can be rotated in a first direction to engage with a feature of the housing 304 to impede axial movement of the slider assembly 308 within the housing 304, which can lock the relative positioning of the tube 306 relative to the elongate element 314. The lock 312 can be rotated in a second direction, which can be opposite the first direction, to disengage with the feature of the housing 304 to permit axial movement of the slider assembly 308 within the housing 304. A user, such as a clinician (e.g., surgeon), can move the slide 310 with the lock 312 disengaged from the feature of the housing 304 to move the tube 306 relative to the housing 304.

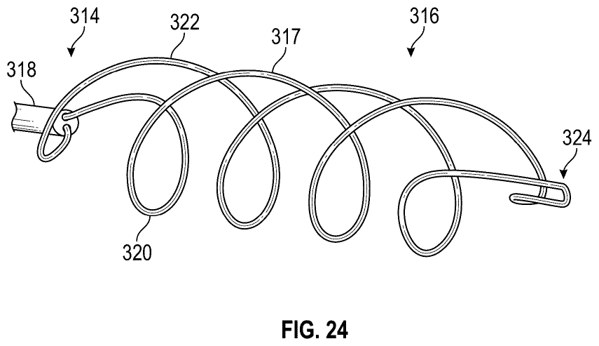

FIG. 24 illustrates an enlarged view of the distal portion 318 of the elongate element 314. As shown, the distal portion 318 can include the expandable member 316. The expandable member 316, in some variants, can be self-expanding. For example, with the expandable member 316 uncovered by the tube 306, the expandable member 316 can assume an expanded configuration. The expandable member 316 can include a shape memory material, such as a nickel titanium alloy (e.g., Nitinol). The expandable member 316 can assume a collapsed configuration with the tube 306 covering the expandable member 316. The expandable member 316 can expand to contact a wall of a vein, which can include adjusting a diameter of the expandable member 316 as the inner diameter of the vein changes. The expandable member 316 can expand to varying degrees to contact a changing inner diameter of the vein, which can including changing as the expandable member 316 is proximally retracted through vein to damage a length of the vein.

The expandable member 316 can include one or more coils, which can include a double helix and/or double coiled configuration. For example, the expandable member 316 can include a wire 317, which can be referred to as an elongate conductor, conductor, and/or heated element. The wire 317 can include multiple wires coupled together. The wire 317 can include a first portion 320 and/or a second portion 322. The first portion 320 and second portion 322 can be two wires that are coupled together to form the wire 317. The first portion 320 and second portion 322 can be portions of the same wire that form the wire 317. The first portion 320 can be a positive portion of the wire 317, which can include carrying energy (e.g., current, direct current, etc.) distally. The second portion 322 can be a negative portion of the wire 317, which can include carrying energy (e.g., current, direct current, etc.) proximally back. The first portion 320 can be coiled. The second portion 322 can be coiled. The first portion 320 and second portion 322 can be arranged such that the coils of the first portion 320 and second portion 322 wrap around the same axis. The coils of the first portion 320 can be disposed between the coils of the second portion 322, which can include being centrally positioned between coils of the second portion 322. The first portion 320 and second portion 322 can join at a distal tip 324. The distal tip 324 can include a loop formed by the first portion 320 and second portion 322 that extends distally.

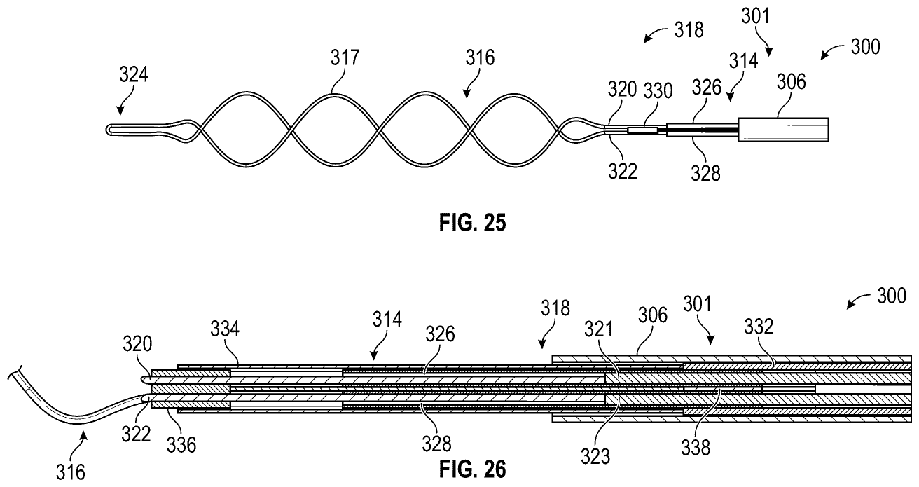

FIG. 25 illustrates a side view of the distal portion 318 of the elongate element 314. As illustrated, the distal portion 318 can include a temperature sensor 330, which can be a thermocouple, thermistor, or the like. The temperature sensor 330 can be disposed proximate the expandable member 316, which can include being disposed proximate (e.g., in contact with) the wire 317 (e.g., first portion 320 and/or second portion 322). In some variants, the temperature sensor 330 can be coupled to one or both of the first portion 320 and/or second portion 322. For example, the temperature sensor 330 can be coupled (e.g., heat shrinked) to second portion 322. The temperature sensor 330 can monitor temperature. The user can control the endovenous device 300 based on the sensed temperature. In some variants, the endovenous device 300 can automatically control energy provided to the expandable member 316 based on the sensed temperature, which can include providing pulse-width modulation of the energy.

The wire 317 can be coupled to one or more conductors that provide energy (e.g., electrical, direct current, alternating current, etc.) to the wire 317. For example, as shown in FIG. 26, the elongate element 314 can include a first conductor 321 and/or a second conductor 323. The first portion 320 of the wire 317 can be coupled to the first conductor 321 (e.g., positive conductor), which can be a copper wire (e.g., positive copper wire). The second portion 322 of the wire 317 can be coupled to the second conductor 323 (e.g., negative conductor), which can be a copper wire (e.g., negative copper wire). A crimp 326 can couple the first portion 320 to the first conductor 321. A crimp 328 can couple the second portion 322 to the second conductor 323. The first conductor 321 can deliver energy to the expandable member 316 from the handle 172. The second conductor 323 can carry energy back to the handle 172 from the expandable member 316. The first conductor 321 can deliver energy to the expandable member 316 and the second conductor 323 can carry energy proximally away from the expandable member 316.

The elongate element 314 can include a tube 332, which can be referred to as an inner tube, catheter, and/or inner catheter. The tube 332 can be disposed within the tube 306. The first conductor 321 and second conductor 323 can be disposed through the tube 332. The expandable member 316 (e.g., wire 317) can be coupled to the tube 332. For example, a covering 334, such as heat shrink, can be disposed over a proximal portion of the wire 317 (e.g., first portion 320 and/or second portion 322) and a distal portion of the tube 332.

The elongate element 314 can include adhesive 336, which can include a structure formed of adhesive (e.g., liquid adhesive that has set) such as an adhesive cylinder. The adhesive 336 can be disposed on proximal portions of the wire 317 (e.g., first portion 320 and second portion 322). The adhesive 336 can hold the proximal portions of the wire 317 (e.g., first portion 320 and second portion 322) in a desired configuration. For example, the adhesive 336 can hold the proximal portions of the wire 317 (e.g., first portion 320 and second portion 322) close together but not touching. The covering 334 can be disposed over the adhesive 336, which can include being disposed over a proximal portion of the adhesive 336. In some variants, the adhesive 336 can protrude distally out of the covering 334.

The elongate element 314 can include adhesive 338, which can include a structure formed of adhesive (e.g., liquid adhesive that has set) such as an adhesive cylinder. The adhesive 338 can be disposed on distal portions of the first conductor 321 and second conductor 323. The adhesive 338 can hold the distal portions of the first conductor 321 and second conductor 323 in a desired configuration. For example, the adhesive 338 can hold the distal portions of the first conductor 321 and second conductor 323 close together but not touching. The adhesive 338 can be adhered to an interior of the tube 332. In some variants, the adhesive 338 can be disposed over portions of the crimps 326, 328 disposed on the first conductor 321 and second conductor 323. The adhesive 338 can, in some variants, help to maintain the positioning of the first conductor 321 and second conductor 323 within the tube 332.

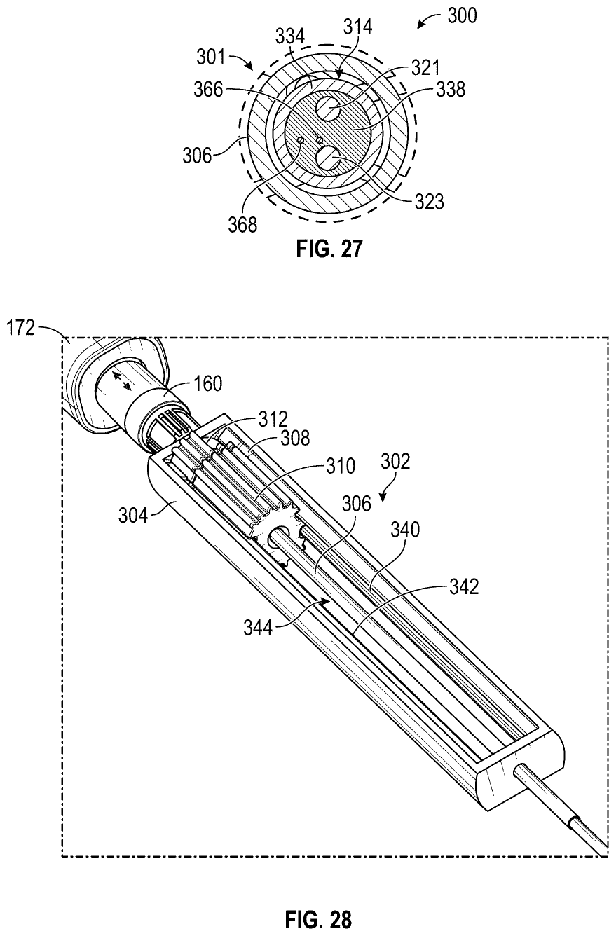

FIG. 27 illustrates a section view of the distal portion 318 of the elongate element 314 disposed through the tube 306. The section view is on a plane perpendicular to the longitudinal axis of the elongate element 314 and tube 306. As shown, the elongate element 314 can include one or more conductors (e.g., wires) for the temperature sensor 330. The one or more wires can provide energy (e.g., electrical, direct current, alternating current) to the temperature sensor 330 for operation. The one or more wires can facilitate the communication of data (e.g., temperature data) from the temperature sensor 330 to the handle 172 or another device in communication with the handle 172. For example, the temperature sensor 330 can include a positive temperature sensor wire 366 and/or a negative temperature sensor wire 368.

FIG. 28 illustrates the actuator 302. The actuator 302 can be coupled to the connector 160 of the handle 172, which can be a corresponding connector. As described herein, the actuator 302 can include the housing 304. The housing 304 can include an interior 344, which can be referred to as a channel and/or slot. The interior 344 of the housing 304 can include rail 340 and/or rail 342, which can be referred to as guides and/or tracks. The rail 340 and/or rail 342 can be disposed along opposite interior walls of the housing 304. The rail 340 and/or rail 342 can extend longitudinally in a direction that is parallel with a central longitudinal axis of the housing 304. The slider assembly 308 can be axially translated (e.g., slid) in the interior 344. The slider assembly 308 can interface with the rail 340 and/or rail 342 to guide linear movement of the slider assembly 308 in the interior 344. The slider assembly 308 can interface with the rail 340 and/or rail 342 to restrict movement other than linear movement along the longitudinal axis of the housing 304. The slider assembly 308 can include features that interface with (e.g., are inserted into) the rails 340, 342.

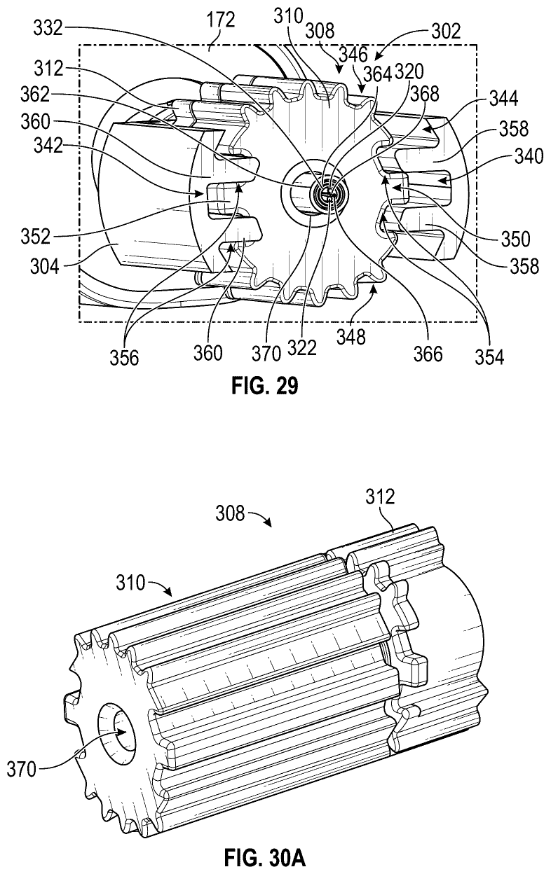

For example, as shown in FIG. 29, the rail 340 can include protrusions 358 and/or the rail 342 can include protrusions 360. The protrusions 358, 360 can extend inward from the wall of the housing 304. The slide 310 can include recesses 354 (e.g., cutouts, grooves, channels) and/or recesses 356 (e.g., cutouts, grooves, channels). The slide 310 can include protrusions 350, 352. The protrusions 350, 352 can be disposed one hundred and eighty degrees away from each other about the central axis of the slide 310. The protrusion 350 can be disposed between protrusions 358. The protrusion 352 can be disposed between protrusions 360. The recesses 354 of the slide 310 can receive the protrusions 358 of the housing 304. The recesses 356 of the slide 310 can receive the protrusions 360 of the housing 304. As the slider assembly 308 is linearly advanced and retracted within the interior 344 the housing 304, the protrusions 358 can slide in the recesses 354, the protrusion 350 can slide between the protrusions 358, the protrusions 360 can slide in the recesses 356, and/or the protrusion 352 can slide between the protrusions 360.

The housing 304 can include top and/or bottom openings that can enable a user to access (e.g., push, pull, grasp, touch) the slider assembly 308, which can include the slide 310 and/or lock 312, to move the slider assembly 308 within the housing 304. The slide 310 can include features to facilitate handling of the slide 310. For example, the slide 310 can include handling feature(s) 346, which can include texture(s), ridge(s), flange(s), protrusion(s), bump(s), and/or other features. The handling feature(s) 346 can be accessible via the top opening of the housing 304, which can include the handling feature(s) 346 protruding out of the top opening of the housing 304. The slide 310 can include handling feature(s) 348, which can include texture(s), ridge(s), flange(s), protrusion(s), bump(s), and/or other features. The handling feature(s) 348 can be accessible via the bottom opening of the housing 304, which can include the handling feature(s) 348 protruding out of the bottom opening of the housing 304.

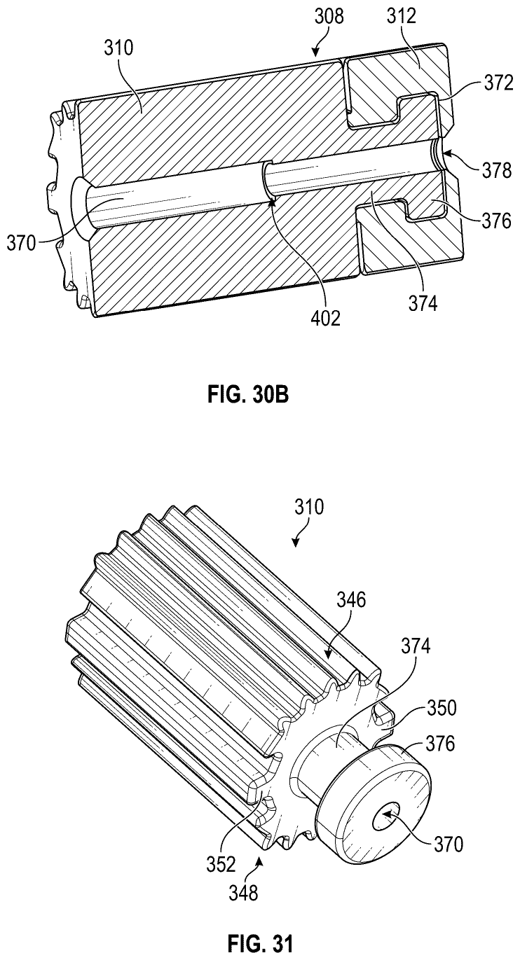

FIG. 30A illustrates a view of the slider assembly 308 outside the housing 304. As shown, the slider assembly 308 can have a generally cylindrical shape. As described herein, the slide 310 and lock 312 can be rotatably coupled together. For example, as shown in FIG. 30B, the lock 312 can include an interior region 372 (e.g., pocket, cavity, hole). The slide 310 can include a protrusion 374 that can be disposed within the interior region 372 to rotatably couple the slide 310 and lock 312 together. The shape and size of the interior region 372 can correspond to that of the protrusion 374. In some variants, the protrusion 374 can include an enlarged end portion 376 that can impede removal of the protrusion 374 from the interior region 372—for example, the enlarged end portion 376 can include a periphery larger than that of a portion of the interior region 372 through which the enlarged end portion 376 would pass if removed from the interior region 372.

The slide 310 can include a passage 370 (e.g., bore, duct) through which one or more tubes, wires, shafts, and/or other features can pass. The lock 312 can include a hole 378 (e.g., opening) through which one or more tubes, wires, shafts, and/or other features can pass. The passage 370 and hole 378 can be coaxially aligned, as shown in FIG. 30B. The passage 370 can include a ledge 402 (e.g., shelf, lip). The ledge 402 can contact a tube received into the passage 370 that is advanced and retracted with the slide 310, which can include the tube 306 and/or a tube coupled to the tube 306.

FIG. 31 illustrates the slide 310 decoupled from the lock 312. The slide 310 can have a generally cylindrical shape. The protrusion 374 can extend from an end (e.g., proximal end) of the generally cylindrical shape. The passage protrusion 374 can be disposed at an end of the slide 310.

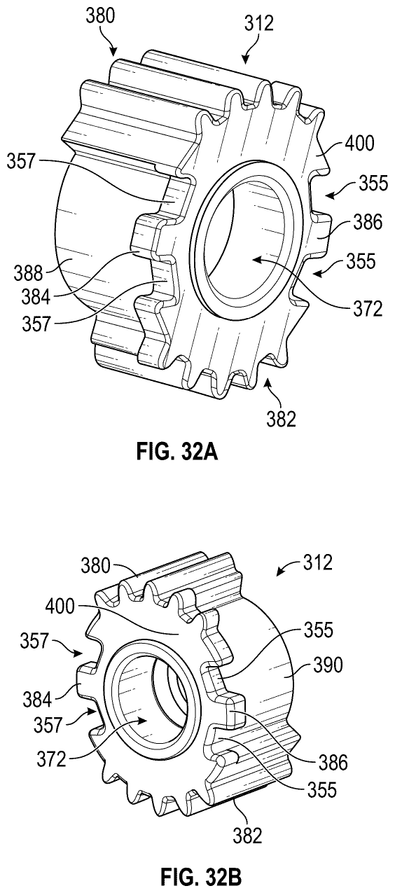

FIGS. 32A and 32B illustrate the lock 312 decoupled from the slide 310. The lock 312 can include a generally cylindrical shape. The lock 312 can be shorter than the slide 310. The lock 312 can be less than half, one third, or one fourth of a length of the slide 310. The lock 312 can include one or more handling features to facilitate handling (e.g., grasping, rotating, spinning). For example, the lock 312 can include handling feature(s) 380, which can include texture(s), ridge(s), flange(s), protrusion(s), bump(s), and/or other features. The handling feature(s) 380 can be accessible via the top opening of the housing 304. In some variants, the handling feature(s) 380 can protrude out of the top opening of the housing 304. The lock 312 can include handling feature(s) 382, which can include texture(s), ridge(s), flange(s), protrusion(s), bump(s), and/or other features. The handling feature(s) 382 can be accessible via the bottom opening of the housing 304. In some variants, the handling feature(s) 382 can protrude out of the bottom opening of the housing 304. The lock 312 can include a gap 388, which can be a circumferential gap. The lock 312 can include a gap 390, which can be a circumferential gap. The gap 388 can be disposed circumferentially between the handling feature(s) 380 and the handling feature(s) 382. The gap 390 can be disposed circumferentially between the handling feature(s) 380 and the handling feature(s) 382, which can include being disposed on an opposite side of the lock 312 relative to the gap 388. The gap 388 and/or gap 390 can define regions of reduced radial size compared to the handling feature(s) 380 and/or handling feature(s) 382.

The lock 312 can include one or more protrusions that can be aligned to interface with the rail 340 and/or rail 342 to allow the slider assembly 308 to be advanced in the housing 304. When not aligned to interface with the rail 340 and/or rail 342, the one or more protrusions can impede advancement of the slider assembly 308 in the housing 304. For example, the lock 312 can include a portion 400 (e.g., flange, segment). The portion 400 can include a protrusion 384 (e.g., tab), protrusion 386 (e.g., tab), recesses 355 (e.g., cutouts, notches), and/or recesses 357 (e.g., cutouts, notches). The protrusions 384, 386 can be disposed one hundred and eighty degrees away from each other. The protrusion 384 can be disposed between the recesses 357. The protrusion 386 can be disposed between the recesses 355. The portion 400 can include a periphery that matches that of the slide 310. The portion 400 can be disposed at an end (e.g., distal end) of the lock 312. The portion 400 can be disposed at the end of the lock 312 that receives the protrusion 374.

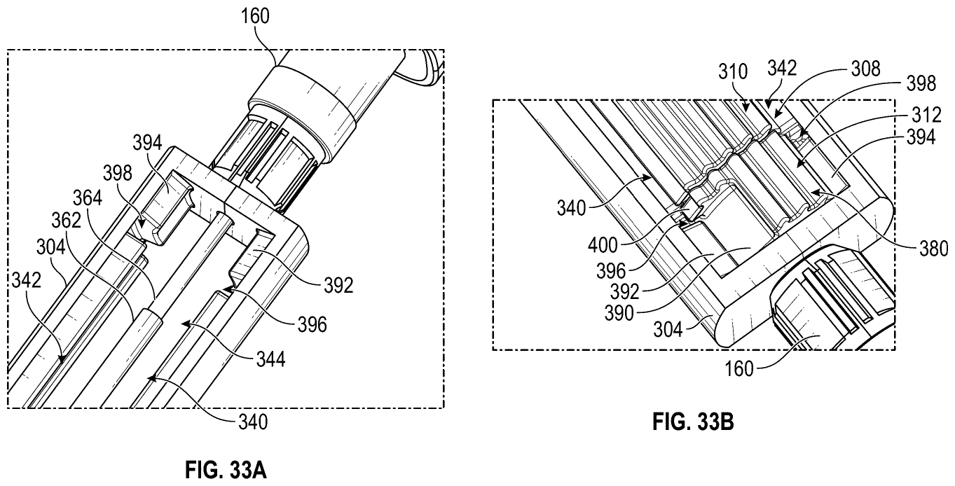

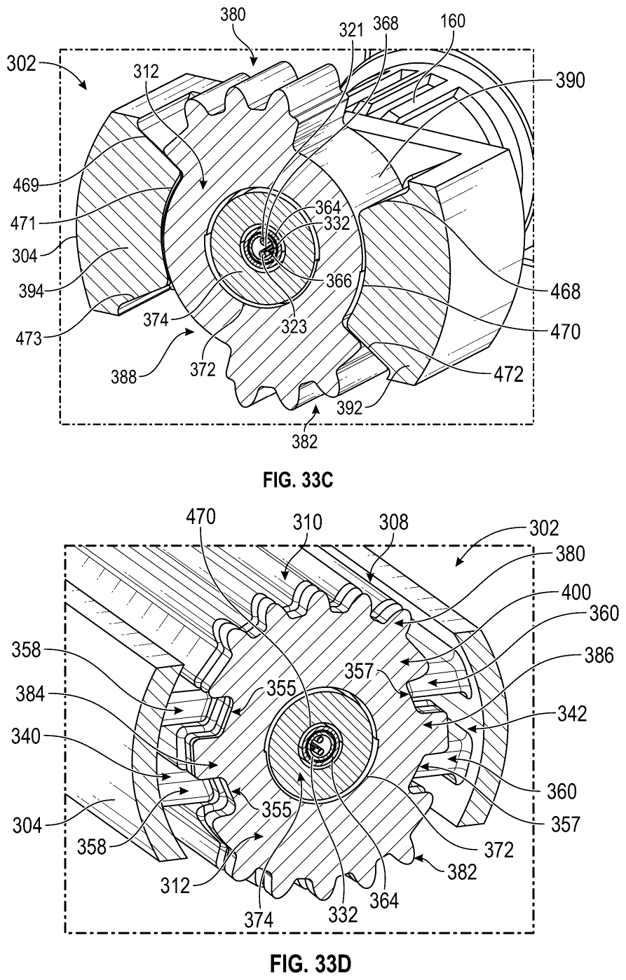

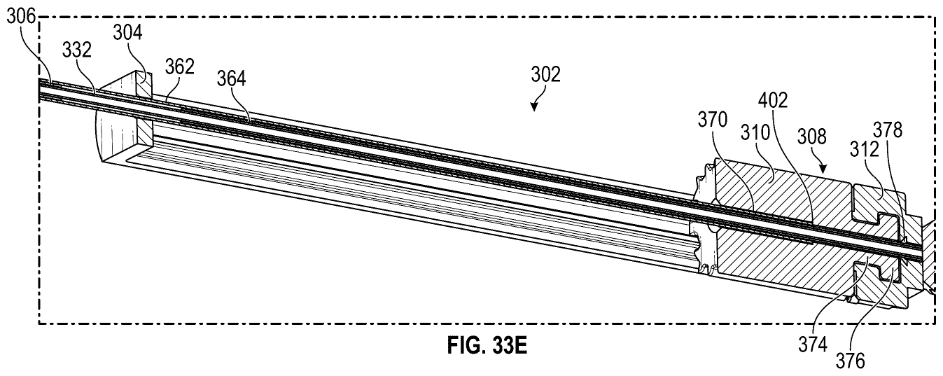

FIG. 33A illustrates the housing 304 without the slider assembly 308 disposed therein. One or more tubes, wires, shafts, and/or other features can extend through the interior 344 of housing 304. As shown, the outer support tube 362 and inner support tube 364 can extend through the interior 344.

The housing 304 can include one or more features to limit the rotational movement of the lock 312 in the housing 304 when rotating between locked and unlocked configurations. For example, the housing 304 can include a protrusion 392 and/or protrusion 394. The protrusion 392 and/or protrusion 394 can extend inward toward the interior 344 from the interior walls of the housing 304 to limit the rotational movement of the lock 312 in the housing 304 when rotating between locked and unlocked configurations. The housing 304 can include a gap 396 and/or gap 398. The gap 396 can be disposed between the protrusion 392 and the rail 340. The gap 398 can be disposed between the protrusion 394 and the rail 342. As illustrated in FIG. 33B, the portion 400 can be disposed in the gap 396 and/or gap 398 to facilitate rotation of the lock 312 within the interior 344 of the housing 304.

FIG. 33C illustrates a section view of the lock 312 in the housing 304. As illustrated, the protrusion 392 can include an upper surface 468, inward-facing surface 470, and/or lower surface 472. The protrusion 394 can include an upper surface 469, inward-facing surface 471, and/or lower surface 473. In some variants, the protrusion 392 and protrusion 394 can be in a mirrored arrangement relative to a central longitudinal plane of the housing 304. The upper surface 468 and/or upper surface 469 can be angled. The inward-facing surface 470 and/or inward-facing surface 471 can be curved, which can include being curved to correspond to a periphery of the lock 312 along the gap 388 and/or gap 390. The lower surface 472 and/or lower surface 473 can be angled. The protrusion 392 can be disposed in the gap 390 of the lock 312. The protrusion 394 can be disposed in the gap 388 of the lock 312. The lock 312 can be rotated such that the handling feature(s) 380 move between contacting the upper surface 469 of the protrusion 394 and contacting the upper surface 468 of the protrusion 392. The lock 312 can be rotated such that the feature(s) 382 move between contacting the lower surface 472 of the protrusion 392 and contacting the lower surface 473 of the protrusion 394.

As described herein, one or more features, such as tubes, wires, and/or shafts, can be routed through the slider assembly 308 (e.g., slide 310 and lock 312). For example, the tube 332 and/or inner support tube 364 can be routed through the passage 370 of the slide 310, which can include through the protrusion 374 of the slide 310. The first conductor 321, second conductor 323, positive temperature sensor wire 366, and/or negative temperature sensor wire 368 can be routed through the tube 332. The lock 312 can rotate relative to the slide 310 (e.g., protrusion). The lock 312 can rotate relative to the tube 332 and/or inner support tube 364.

FIG. 33D illustrates a section view of the lock 312 in the housing 304 in an unlocked configuration. In the unlocked configuration, the periphery of the portion 400 of the lock 312 can be aligned with the periphery of the slide 310. In the unlocked configuration, the protrusion 384 of the lock 312 can be aligned between the protrusions 358 of the housing 304, the recesses 355 of the lock 312 can be aligned with the protrusions 358 of the housing 304, the protrusion 386 of the lock 312 can be aligned between the protrusions 360 of the housing 304, and/or the recesses 357 of the lock 312 can be aligned with the protrusions 360 of the housing 304. In the unlocked configuration, the slider assembly 308 can be advanced such that the protrusion 384 of the lock 312 is between the protrusions 358 of the housing 304, the protrusions 358 of the housing 304 are disposed in the recesses 355 of the lock 312, the protrusion 386 of the lock 312 is between the protrusions 360 of the housing 304, and/or the protrusions 360 of the housing 304 are disposed in the recesses 357 of the lock 312. In the unlocked configuration, the slider assembly 308 can be advanced and retracted in the housing 304 but rotation of the lock 312 can be restricted when advanced such that the portion 400 is distal of the gaps 396, 398. In some variants, rotating the lock 312 in a first direction (e.g., clockwise) can place the lock 312 in the unlocked configuration shown in FIG. 33D, which can include rotating the handling feature(s) 380 to contact the upper surface 469 of the protrusion 394 of the housing 304 and the handling feature(s) 382 to contact the lower surface 472 of the protrusion 392 of the housing 304. The advancement and retraction can move, directly or indirectly, the tube 306 to cover and uncover the expandable member 316. Advancing the tube 306 to cover the expandable member 316 can cause the expandable member 316 to collapse. Retracting the tube 306 to uncover the expandable member 316 can allow the expandable member 316 to self-expand.

In some variants, rotating the lock 312 in a second direction (e.g., counterclockwise), which can be opposite the first direction, can place the lock 312 in the locked configuration that impedes advancement of the slider assembly 308 in the housing 304, which can include rotating the handling feature(s) 380 to contact the upper surface 468 of the protrusion 392 of the housing 304 and the handling feature(s) 382 to contact the lower surface 473 of the protrusion 394 of the housing 304. In the locked configuration, the protrusion 384 of the lock 312 may not be aligned between the protrusions 358 of the housing 304 and/or the protrusion 386 of the lock 312 may not be aligned between the protrusions 360 of the housing 304, which can physically impede advancement of the slider assembly 308 as other portions of the portion 400 contact the protrusions 358 and/or protrusions 360.

In some variants, the housing 304 can include multiple gaps, similar to the gaps 398, 396, distributed along a length of the interior 344 of the housing 304, which can enable the lock 312 to be rotated to a locked configuration at different locations along the length of the housing 304 to lock the tube 306 at different positions relative to the expandable member 316. For example, in some variants, the tube 306 can be locked at a position that covers or uncovers the expandable member 316.

FIG. 33E illustrates a section view of the actuator 302 and one or more tubes of the ablation assembly 301 of the endovenous device 300. As shown, the ablation assembly 301 can include an outer support tube 362, tube 306, inner support tube 364, and/or tube 332. In some variants, the outer support tube 362, inner support tube 364, and/or tube 332 can extend through the housing 304. In some variants, the tube 332 and inner support tube 364 can extend through the slider assembly 308. The outer support tube 362 can be coupled to the slide 310 of the slider assembly 308. For example, the outer support tube 362 can contact the ledge 402 disposed in the passage 370 of the slide 310. In some variants, the outer support tube 362 can be adhered to the wall of the passage 370. The tube 306 can be coupled (e.g., adhered) to the outer support tube 362. In some variants, a distal portion of the outer support tube 362 can be coupled to the tube 306. In some variants, a proximal portion of the tube 306 can be disposed within a distal portion of the outer support tube 362. In some variants, a proximal portion of the tube 306 can receive therein a distal portion of the outer support tube 362. The outer support tube 362 can have greater rigidity than the tube 306. For example, the outer support tube 362 can include a more rigid material (e.g., metal such as stainless steel) and the tube 306 can include a less rigid material (e.g., flexible polymer). The outer support tube 362 can facilitate sliding through the housing 304. The tube 332 can extend through the inner support tube 364. In some variants, the inner support tube 364 can have a greater rigidity than the tube 332. The inner support tube 364 and/or tube 332 can remain in fixed axial positions relative to the housing 304. The inner support tube 364 can facilitate sliding of the slider assembly 308 and/or outer support tube 362 over the inner support tube 364 and tube 332. The inner support tube 364 can include a more rigid material (e.g., metal such as stainless steel) and the tube 332 can include a less rigid material (e.g., flexible polymer). The inner support tube 364 can include a length that terminates prior to exiting the housing 304. In some variants, the inner support tube 364 and/or tube 332 can be coupled to the housing 304, which can include being fixedly coupled thereto. The inner support tube 364 and tube 332, in some variants, can be coupled (e.g., adhered) together. In use, the outer support tube 362 and/or inner support tube 364 may not be configured to be inserted into a patient.

In use, the ablation assembly 301 can percutaneously access the vasculature of a patient, such as a human patient. The ablation assembly 301 can be navigated through the vasculature to a target site, which can be a varicose vein (e.g., varicose vein of the leg). When navigating through the vasculature, the expandable member 316 can be in a collapsed configuration. For example, the tube 306 can cover the expandable member 316 to place the expandable member 316 in the collapsed configuration. The tube 306 can be advanced by rotating the lock 312 of the slider assembly 308 to the unlocked configuration and distally advancing the slider assembly 308 within the housing 304 to cause the tube 306 to cover the expandable member 316 and place the expandable member 316 in a collapsed configuration. With the distal portion of the ablation assembly 301 at the target site, the tube 306 can be retracted to uncover the expandable member 316. For example, the slider assembly 308 can be translated proximally in the housing 304 to unsheathe the expandable member 316. The slider assembly 308 can be moved to the locked configuration in the housing 304 as described herein. The uncovered expandable member 316 can self-expand to an expanded configuration, which can include self-expanding to contact the vein wall. The user can command the handle 172 to deliver energy (e.g., electrical, direct current, alternating current, radio frequency, non-radio frequency) to the expandable member 316 to ablate the vein wall, which can damage the vein wall. In some variants, energy can be supplied by one or more power sources (e.g., battery, generator, power connection, wall outlet, etc.). In some variants, direct current can resistively heat the expandable member 316 to ablate the vein wall. In some variants, the handle 172 can deliver energy to the expandable member 316 in pulses. In some variants, the handle 172 and/or a device in communication with the handle 172 can automatically adjust the energy delivery based on temperatures sensed by the temperature sensor 330. In some variants, the expandable member 316 can be moved (e.g., rotated and/or translated within the vein), automatically and/or manually, to mechanically damage the vein wall. For example, in some variants, the user can move the handle 172 distally and/or proximally to translate the expandable member 316 within the vein to damage a length of the vein wall with thermal energy and/or mechanical abrasion. The ablation assembly 301 can be proximally retracted along a length of the vein to damage the length of the vein. The damage to the vein wall can cause the vein to collapse, which can close off the vein and ultimately result in the vein being reabsorbed into the body. With ablation complete at the target site, the expandable member 316 can be collapsed for removal of the ablation assembly 301. For example, the lock 312 of the slider assembly 308 can be rotated from the locked configuration to the unlocked configuration such that the user can advance the slider assembly 308 distally in the housing 304 to distally advance the tube 306 to cover the expandable member 316 and place the expandable member 316 in a collapsed configuration. With the expandable member 316 collapsed, the ablation assembly 301 can be proximally retracted out of the vasculature of the patient.

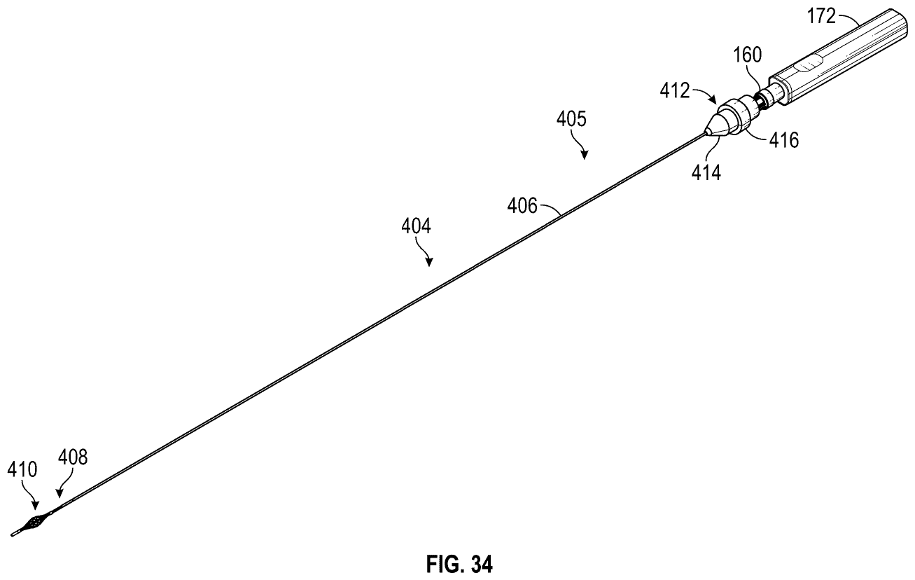

FIG. 34 illustrates an endovenous device 404, which can also be referred to as an ablation device. The endovenous device 404 can be used to treat varicose veins. The endovenous device 404 can include an ablation assembly 405, which can also be referred to as an ablation catheter and/or ablation catheter assembly. The ablation assembly 405 can be coupled to the handle 172. For example, the ablation assembly 405 can be coupled to the handle 172 by way of the connector 160. The handle 172 can provide energy (e.g., electrical, direct current, alternating current, radio frequency, non-radio frequency) to the ablation assembly 405 to ablate.

The ablation assembly 405 can include an elongate element 408, which can also be referred to as an elongate member, catheter, and/or inner catheter. The elongate element 408 can include an expandable member 410 (e.g., self-expanding member, heated element). The expandable member 410 can be disposed at a distal portion of the elongate element 408.

The elongate element 408 can include a tube 406 (e.g., outer tube, sheath). The outer tube 406 can be disposed over features of the elongate element 408.

The ablation assembly 405 can include an actuator 412 (e.g., actuation mechanism, actuation device) that can be used to expand and collapse the expandable member 410. For example, the actuator 412 can include a dial 416 (e.g., ring, annular structure, circular band). The dial 416 can be rotated in a first direction to expand the expandable member 410. The dial 416 can be rotated in a second direction, opposite the first direction, to collapse the expandable member 410. The actuator 412 can include a housing 414, which can include a cylindrical shape with a cone disposed on a distal end. The dial 416 can be disposed at a periphery of the housing 414, which can include being disposed at a cylindrical portion of the housing 414. The outer tube 406 can be coupled (e.g., adhered) to the housing 414, which can include a distal portion (e.g., conical portion) of the housing 414.

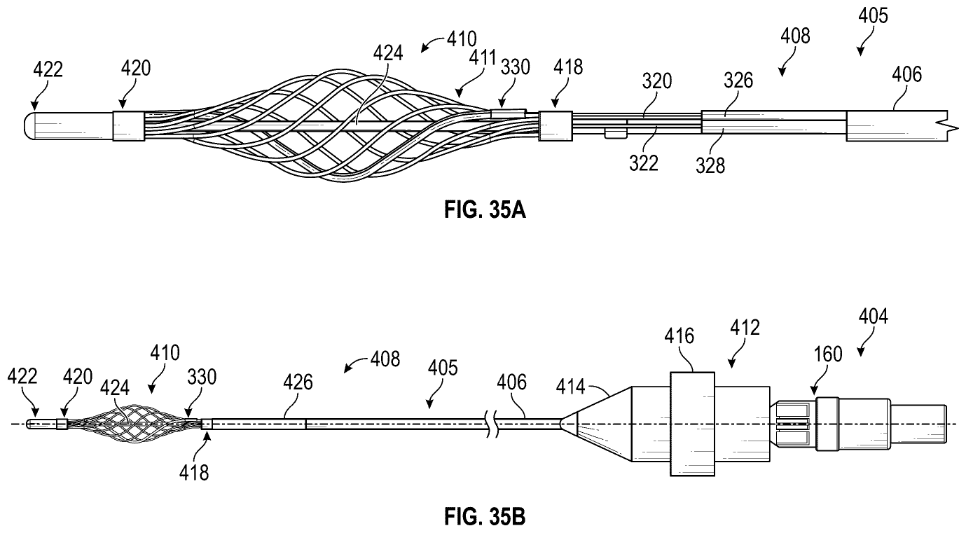

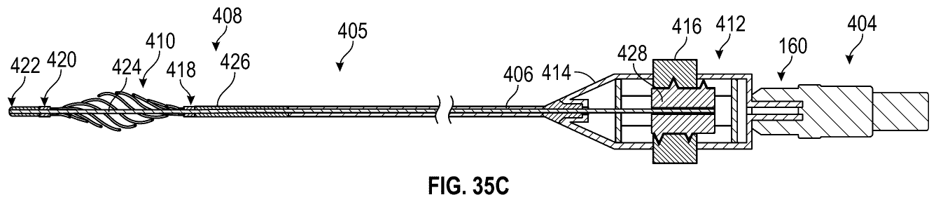

FIG. 35A illustrates a distal portion of the elongate element 408 showing the expandable member 410. As described, the expandable member 410 can be positioned in a collapsed configuration and an expanded configuration. In the expanded configuration, the expandable member 410 can have a proximal tapered portion, distal tapered portion, and enlarged middle portion as illustrated. In the expanded configuration, the expandable member 410 can have a spindle and/or fusiform shape. In the collapsed configuration, the expandable member 410 can have a generally cylindrical profile. In the collapsed configuration, the expandable member 410 and elongate element 408 can be disposed in an outer tube, such as a catheter.

As shown, the expandable member 410 can include a plurality of wire portions 411. In some variants, the wire portions 411 can include separate wires. In some variants, the wire portions 411 can include portions of the same wire. In some variants, the expandable member 410 (e.g., wire portions 411) can include a shape memory material (e.g., nickel titanium alloy such as Nitinol). The wire portions 411 can be coupled to a proximal anchor 418 (e.g., annular structure, ring, band, separator). The wire portions 411 can be coupled to a distal anchor 420 (e.g., annular structure, ring, band, separator). The wire portions 411 can span between the proximal anchor 418 and the distal anchor 420.

The elongate element 408 can include a shaft 424 (e.g., wire, tube). The shaft 424 can be a nitinol wire. The shaft 424 can extend through the expandable member 410. The distal anchor 420 can be fixedly coupled to the shaft 424 such that the distal anchor 420 moves with the shaft 424. The shaft 424 can pass through the proximal anchor 418, which can include passing through the proximal anchor 418 without being coupled thereto. The shaft 424 can be distally advanced and proximally retracted relative to (e.g., through) the proximal anchor 418. The wire portions 411 can be wrapped around the shaft 424. For example, the wire portions 411 can be wrapped in a counterclockwise direction around the shaft 424 from a proximal perspective. Each of the wire portions 411 can be coupled to the proximal anchor 418 at an angular position relative to a central longitudinal axis of the shaft 424, wrapped around the shaft 424 (e.g., wrap in a counterclockwise direction), and be coupled to the distal anchor 420 at the same angular position relative to the central longitudinal axis of the shaft 424. In some variants, each of the wire portions 411 can be coiled around the shaft 424 between the proximal anchor 418 and the distal anchor 420. In some variants, each of the wire portions 411 can be spiraled around the shaft 424 between the proximal anchor 418 and the distal anchor 420. To collapse the expandable member 410, the actuator 412 can distally advance the shaft 424, which can move the distal anchor 420 farther away from the proximal anchor 418 in a distal direction and cause the expandable member 410 to collapse inward. To expand the expandable member 410, the actuator 412 can proximally retract the shaft 424, which can move the distal anchor 420 close to the proximal anchor 418 in a proximal direction and cause the expandable member 410 to expand outward. In some variants, the wire portions 411 can be shape set.

The elongate element 408 can include a distal tip 422, which can be atraumatic (e.g., rounded, blunted). The distal tip 422 can be distal to the distal anchor 420. The distal tip 422 can be disposed on the distal end of the shaft 424. The distal tip 422 can be coupled to the shaft 424 and/or distal anchor 420.

The elongate element 408 can include a temperature sensor 330. The temperature sensor 330 can be coupled to the expandable member 410. For example, the temperature sensor 330 can be coupled to (e.g., heat shrinked) to one or more of the wire portions 411. The temperature sensor 330 can detect temperatures at and/or proximate the expandable member 410, which can be used to control the energy delivery to the expandable member 410.

The expandable member 410 can include a first portion 320 (e.g., first conductor portion, first wire portion) and/or second portion 322 (e.g., second conductor portion, second sire portion). The first portion 320 can be a positive conductor that carries energy (e.g., current, direct current, etc.) distally from a first conductor 321. The second portion 322 can be a negative conductor that conducts energy (e.g., current, direct current, etc.) proximally from the expandable member 410 to the second conductor 323. The first conductor 321 can receive energy from a power source as described herein. The first portion 320 can be operatively coupled to the first conductor 321 with crimp 326. The second portion 322 can be operatively coupled to the second conductor 323 with crimp 328. Energy can be delivered to the expandable member 410 to ablate a vein wall. For example, direct current can be delivered to the expandable member 410 to resistively heat the expandable member 410 to damage (e.g., ablate) the vein wall. The wire portions 411 can be coupled together to facilitate the flow of electrical energy therethrough. In some variants, one or more of the wire portions 411 can carry energy distally and one or more of the wire portions 411 can carry energy back proximally. In some variants, the proximal anchor 418 can include one or more conductors to carry energy between different wire portions 411. In some variants, the distal anchor 420 can include one or more conductors to carry energy between different wire portions 411. As shown in FIG. 35B, the elongate element 408 can include an adhesive 426 (e.g., adhesive structure) coupling the proximal anchor 418 to the outer tube 406. In some variants, the proximal anchor 418, adhesive 426, outer tube 406, and housing 414 are fixedly coupled together. In some variants, the outer tube 406 is adhered to a distal portion of the housing 414.

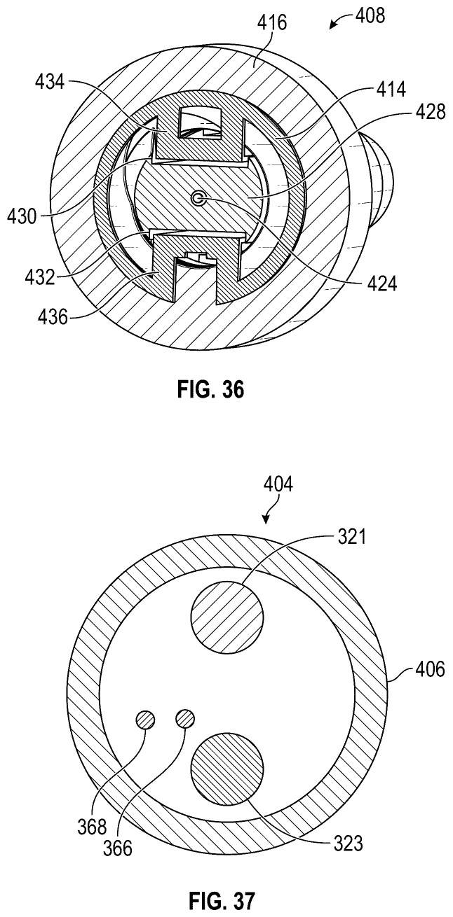

FIG. 35C illustrates a section view of the endovenous device 404. As shown, the shaft 424 can extend through the outer tube 406 to an interior of the housing 414. The shaft 424 can be coupled (e.g., fixedly coupled, adhered) to a threaded shaft 428. The threaded shaft 428 can be disposed inside the housing 414. The threaded shaft 428 can include external threads. The dial 416 can be interfaced with the threaded shaft 428. The dial 416 can include internal threads that are engaged with the external threads of the threaded shaft 428. As shown in FIG. 36, the threaded shaft 428 can include a channel 430 (e.g., upper channel, slot, groove, recess) and/or channel 432 (e.g., lower channel, slot, groove, recess). The channel 430 and channel 432 can be disposed on opposite sides of the threaded shaft 428 (e.g., one hundred and eighty degrees apart from each other). The housing 414 can include a protrusion 434 and/or protrusion 436. The protrusion 434 and/or protrusion 436 can extend inward. The protrusion 434 can be disposed in the channel 430. The protrusion 436 can be disposed in the channel 432. The interface between the protrusion 434 and channel 430 can impede rotation of the threaded shaft 428 as the dial 416 is rotated such that rotational movement of the dial 416 causes distal advancement or proximal retraction of the threaded shaft 428 and the shaft 424 coupled to the threaded shaft 428. The interface between the protrusion 436 and channel 432 can impede rotation of the threaded shaft 428 as the dial 416 is rotated such that rotational movement of the dial 416 causes distal advancement or proximal retraction of the threaded shaft 428 and the shaft 424 coupled to the threaded shaft 428.

FIG. 37 illustrates a section view of the ablation assembly 405 through the tube 406. The section view is on a plane perpendicular to the longitudinal axis of the tube 406. As shown, the first conductor 321, second conductor 323, positive temperature sensor wire 366, and/or negative temperature sensor wire 368 can be disposed through the interior of the outer tube 406.

In use, the ablation assembly 405 can percutaneously access the vasculature of a patient, such as a human patient. The ablation assembly 405 can be navigated through the vasculature to a target site, which can be a varicose vein (e.g., varicose vein of the leg). When navigating through the vasculature, the expandable member 410 can be in a collapsed configuration. For example, the dial 416 can be rotated in the second direction to distally advance the shaft 424 and/or distal tip 422 relative to the proximal anchor 418 and/or outer tube 406 to collapse the expandable member 410. In some variants, the expandable member 410 can be covered via an outer tube when being navigated to the target site, unsheathed when at the target site to expand and ablate, and covered again via the outer tube when being proximally retracted out of the vasculature. With the expandable member 410 at the target site, the dial 416 can be rotated in the first direction, opposite the second direction, to proximally retract the shaft 424 and distal tip 422 relative to the proximal anchor 418 and/or outer tube 406 to cause the expandable member 410 to expand outward. The expandable member 410 can be expanded to different diameters as appropriate (e.g., more rotation for proximal retraction of the shaft 424 can result in more expansion). The expandable member 410 can be expanded to contact the vein wall. The user can command the handle 172 to deliver energy (e.g., electrical, direct current, alternating current, radio frequency, non-radio frequency) to the expandable member 410 to ablate the vein wall, which can damage the vein wall. In some variants, energy can be supplied by one or more power sources (e.g., battery, generator, power connection, wall outlet, etc.). In some variants, direct current can resistively heat the expandable member 410 to ablate the vein wall. In some variants, the handle 172 can deliver energy to the expandable member 410 in pulses. In some variants, the handle 172 and/or a device in communication with the handle 172 can automatically adjust the energy delivery based on temperatures sensed by the temperature sensor 330. In some variants, the expandable member 410 can be moved (e.g., rotated and/or translated within the vein), automatically and/or manually, to mechanically damage the vein wall. For example, in some variants, the user can move the handle 172 distally and/or proximally to translate the expandable member 410 within the vein to damage the vein wall. The ablation assembly 405 can be proximally retracted along a length of the vein to damage the length of the vein. The damage to the vein wall can cause the vein to collapse, which can close off the vein and ultimately result in the vein being reabsorbed into the body. With ablation complete at the target site, the expandable member 410 can be collapsed for removal of the ablation assembly 405. For example, the dial 416 can be rotated in the second direction to distally advance the shaft 424 and distal tip 422 relative to the proximal anchor 418 and/or outer tube 406 to collapse the expandable member 410 inward. With the expandable member 410 collapsed, the ablation assembly 405 can be proximally retracted out of the vasculature of the patient. In some variants, the expandable member 410 can be covered (e.g., sheathed) prior to removal.

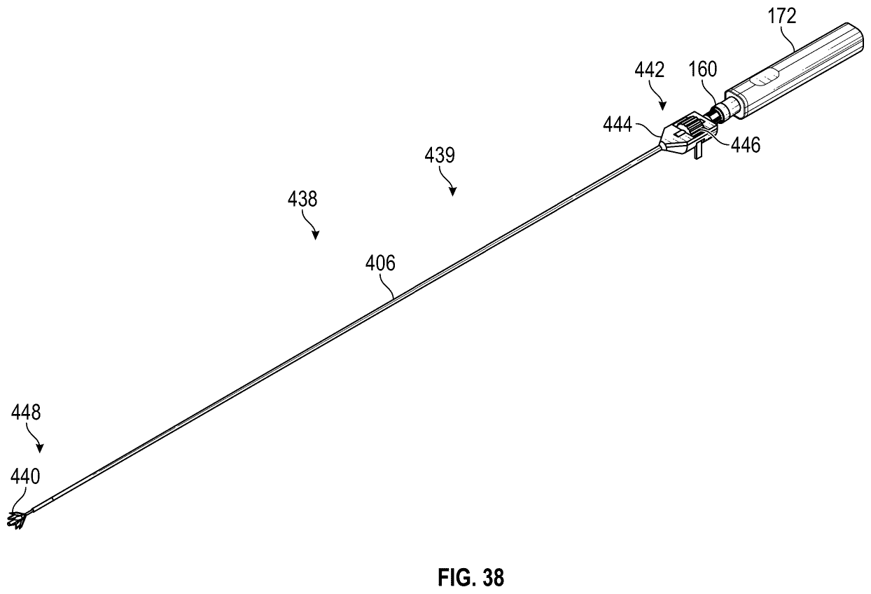

FIG. 38 illustrates an endovenous device 438, which can also be referred to as an ablation device. The endovenous device 438 can be used to treat varicose veins. The endovenous device 438 can include an ablation assembly 439, which can also be referred to as an ablation catheter and/or ablation catheter assembly. The ablation assembly 439 can be coupled to the handle 172. For example, the ablation assembly 439 can be coupled to the handle 172 by way of the connector 160. The handle 172 can provide energy (e.g., electrical, direct current, alternating current, radio frequency, non-radio frequency) to the ablation assembly 439 to ablate.

The ablation assembly 439 can include an elongate element 448, which can also be referred to as an elongate member, catheter, and/or inner catheter. The elongate element 448 can include an expandable member 440 (e.g., self-expanding member, heated element). The expandable member 440 can be disposed at a distal portion of the elongate element 448.

The elongate element 448 can include the tube 406 (e.g., outer tube, sheath). The outer tube 406 can be disposed over features of the elongate element 448.

The ablation assembly 439 can include an actuator 442 (e.g., actuation mechanism, actuation device) that can be used to expand and collapse the expandable member 440. For example, the actuator 442 can include a dial 446 (e.g., disk, cylinder, wheel, annular structure). The dial 446 can be rotated in a first direction to expand the expandable member 440. The dial 446 can be rotated in a second direction, opposite the first direction, to collapse the expandable member 440. The actuator 442 can include a housing 444, which can rotatably support the dial 446. The dial 446 can include gripping features, such as texture, teeth, knobs, ridges, etc., to enable the user to rotate the dial 446. The outer tube 406 can be coupled (e.g., adhered) to the housing 444, which can include a distal portion of the housing 444.

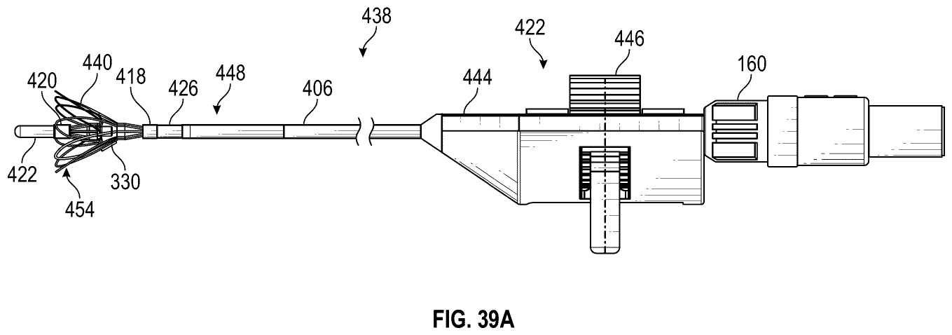

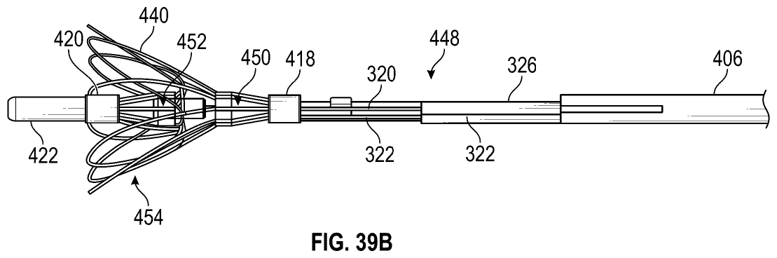

As shown in FIG. 39A, the elongate element 448 can include the proximal anchor 418, distal anchor 420, and/or distal anchor 420. The proximal anchor 418 can be coupled to the outer tube 406, which can include being adhered to the outer tube 406 with adhesive 426 as described herein. The proximal portion of the expandable member 440 can be coupled to the proximal anchor 418 and the distal portion of the expandable member 440 can be coupled to the distal anchor 420.

The expandable member 440 can, in the expanded configuration illustrated in FIG. 39A, have the shape of a cone, which can include an open hollow cone. The expandable member 440 can be tapered, which can include the proximal portion being smaller in diameter than the distal portion. The expandable member 440 can, in the expanded configuration illustrated in FIG. 39A, have the shape of a flower. The expandable member 440 can include one or more wire portions 454 that form loops. The loops, in some variants, can have the appearance of flower pedals such that the expandable member 440 resembles a flower opening when expanding and a flower closing when collapsing.

The elongate element 448 can include the temperature sensor 330, which can include being coupled to the expandable member 440 (e.g., one or more one or more wire portions 454). The temperature sensor 330 can detect temperatures at and/or proximate the expandable member 440.

As illustrated in FIG. 39B, the elongate element 448 can include a proximal insert 450 and/or distal insert 452. The proximal insert 450 can be coupled to the proximal anchor 418. The proximal insert 450 can include a tapered profile that decreases in the proximal direction. The proximal insert 450 can position (e.g., push) a proximal portion of the expandable member 440 radially outward, which can include gradually more outward in the distal direction. Proximal portions of the wire portions 454 can be coupled to the proximal insert 450 and/or proximal anchor 418, which can help maintain separation and/or positioning of proximal portions of the wire portions 454. The distal insert 452 can be coupled to the distal anchor 420. The distal insert 452 can include a tapered profile that decreases in the distal direction. The distal insert 452 can position (e.g., push) a distal portion of the expandable member 440 radially outward, which can include gradually less outward in the distal direction. Distal portions of the wire portions 454 can be coupled to the distal insert 452 and/or distal anchor 420, which can help maintain separation and/or positioning of the distal portions of the wire portions 454.

As shown in FIG. 39B, the expandable member 440 can include a first portion 320 (e.g., first conductor portion, first wire portion) and/or second portion 322 (e.g., second conductor portion, second sire portion). The first portion 320 can be a positive conductor that carries energy (e.g., current, direct current, etc.) distally from a first conductor 321. The second portion 322 can be a negative conductor that delivers energy (e.g., current, direct current, etc.) proximally from the expandable member 440 to the second conductor 323. The first conductor 321 can receive energy from a power source as described herein. The first portion 320 can be operatively coupled to the first conductor 321 with crimp 326. The second portion 322 can be operatively coupled to the second conductor 323 with crimp 328. Energy can be delivered to the expandable member 440 to ablate a vein wall. For example, direct current can be delivered to the expandable member 440 to resistively heat the expandable member 440 to damage (e.g., ablate) the vein wall. The wire portions 454 can be coupled together to facilitate the flow of electrical energy therethrough. In some variants, one or more of the wire portions 454 can carry energy distally and one or more of the wire portions 454 can carry energy back proximally. In some variants, the proximal anchor 418 can include one or more conductors to carry energy between different wire portions 454. In some variants, the distal anchor 420 can include one or more conductors to carry energy between different wire portions 454.

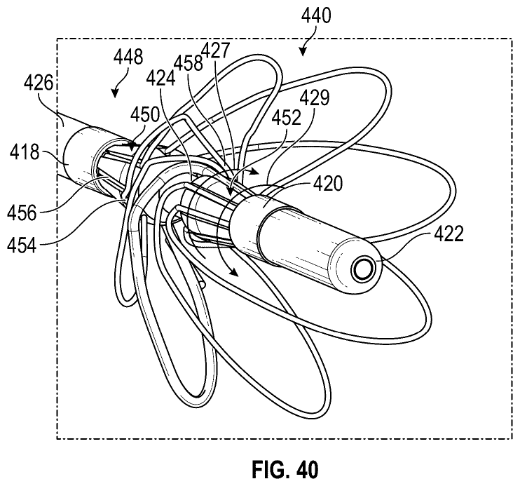

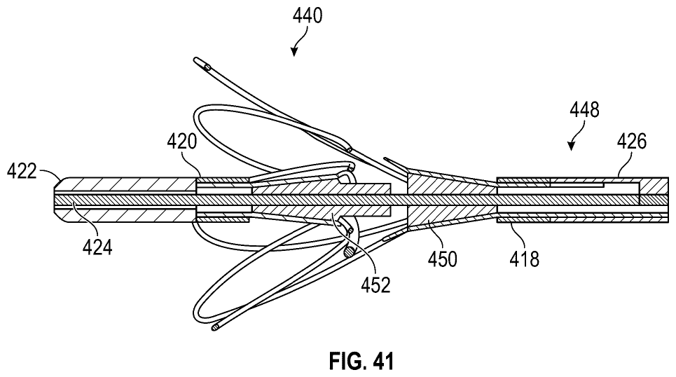

FIGS. 40 and 41 illustrate enlarged views of the expandable member 440 with FIG. 41 showing a section view. As shown, the shaft 424 can extend through the expandable member 440, distal tip 422, proximal anchor 418, distal anchor 420, and/or outer tube 406. The shaft 424 can be fixedly coupled to the distal tip 422 and distal anchor 420. The shaft 424 can rotate relative to the proximal anchor 418 and outer tube 406. When the shaft 424 rotates, the proximal portion of the expandable member 440 can remain rotationally fixed while the distal portion of the expandable member 440 can rotate with the shaft 424, distal anchor 420, and/or distal tip 422. The rotation of the shaft 424 in a first direction can cause the expandable member 440 to expand (e.g., twist to expand) while rotation of the shaft 424 in a second direction, opposite the first direction, can cause the expandable member 440 to collapse. As illustrated in FIG. 40, each of the wire portions 454 can form a loop, which can resemble a flower pedal. Each of the wire portions 454 can include a first end 456 that is coupled to the proximal insert 450 and/or proximal anchor 418 and a second end 458 that is couped to the distal insert 452 and/or distal anchor 420. The second end 458 can be rotationally offset from the first end 456, which can include being 60, 70, 80, 90, 100, 110, 120, 130, 140, 150, 160, 170, 180, 190, 200, or more degrees offset. The wire portions 454 can wrap, coil, and/or spiral around the shaft 424 between the first end 456 and the second end 458 while forming loops between the first end 456 and the second end 458. Each of the wire portions 454 can extend distally (e.g., distally beyond the distal insert 452, distally beyond the proximal end of the distal anchor 420) and radially outward from the proximal anchor 418, curve to extend proximally and radially inward, and then curve to extend distally to couple with the distal anchor 420, which can be at a rotationally offset position as described herein. The rotational (e.g., angular) offset between the first end 456 and second end 458 and the shape of the loop can be configured such that rotating the shaft 424 in the direction of the angular offset between the first end first end 456 and second end 458 (e.g., toward the second end 458), which can be in the direction of arrow 429, can cause the expandable member 440 to expand while rotating the shaft in the opposite direction, which can be in the direction of arrow 427, can cause the expandable member 440 to collapse.

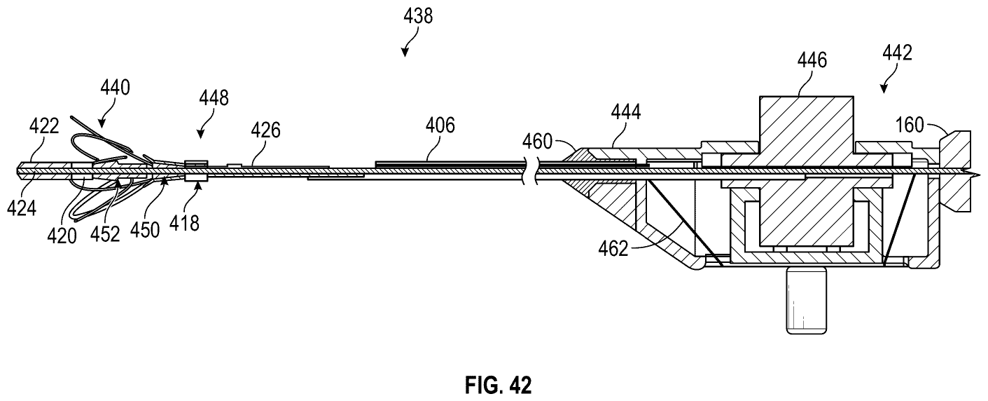

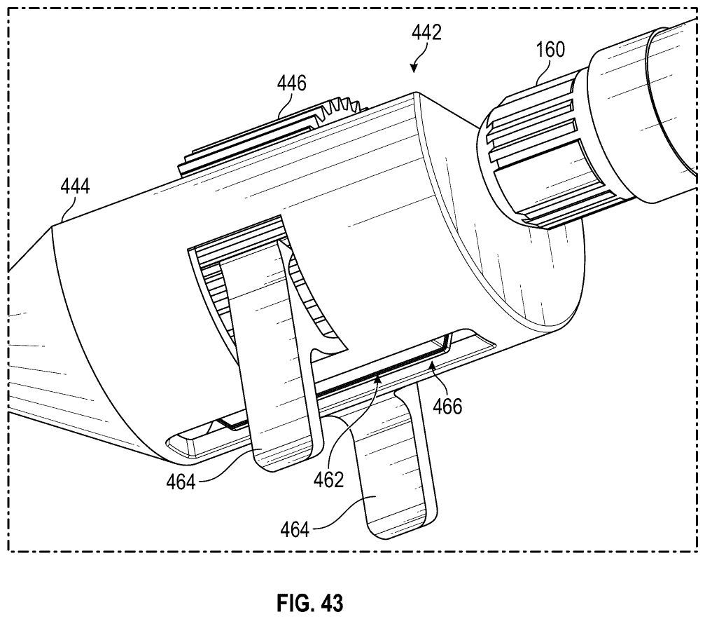

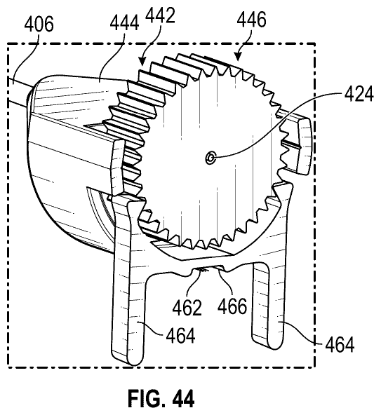

FIG. 42 illustrates a section view of the endovenous device 438. As shown, the shaft 424 can extend through the outer tube 406 to couple (e.g., adhere) to the dial 446. The shaft 424 can rotate with the dial 446. One or more conductors 462, which can include the first conductor 321, second conductor 323, negative temperature sensor wire 368, and/or positive temperature sensor wire 366, can be routed through the outer tube 406 to an interior of the housing 444 and around (e.g., bypass) the dial 446 to be operatively coupled with the connector 160. As illustrated in FIG. 43, the housing 444 can include a channel 466 through which the one or more conductors 462 are routed around the dial 446. The actuator 442 (e.g., housing 444) can include one or more levers 464 that can engage with the dial 446 to impede rotation of the dial 446, which can lock the expandable member 440 in the expanded, collapsed, and/or intermediate configuration (e.g., intermediate being between fully expanded and collapsed). The one or more levers 464 can be biased to engage with the dial 446 to impede rotation of the dial 446 unless moved by the user. As discussed herein, the dial 446 can include external teeth. The one or more levers 464 can include a tooth that can engage with the external teeth to impede rotation of the dial 446.

In use, the ablation assembly 439 can percutaneously access the vasculature of a patient, such as a human patient. The ablation assembly 439 can be navigated through the vasculature to a target site, which can be a varicose vein (e.g., varicose vein of the leg). When navigating through the vasculature, the expandable member 440 can be in a collapsed configuration. For example, after overcoming the biasing force of the one or more levers 464, the dial 446 can be rotated in the second direction to rotate the shaft 424 to collapse the expandable member 440. When collapsed, the one or more levers 464 can be released to lock the dial 446 and the expandable member 440 in the collapsed position. In some variants, the expandable member 440 can be covered via an outer tube when being navigated to the target site, unsheathed when at the target site to expand and ablate, and covered again via the outer tube when being proximally retracted out of the vasculature. With the expandable member 440 at the target site, the dial 446 can be rotated in the first direction, opposite the second direction, to rotate the shaft 424 to cause the expandable member 440 to expand outward. The expandable member 440 can be expanded to different diameters as appropriate depending on the amount of rotation (e.g., more rotation can result in more expansion). The expandable member 440 can be expanded to contact the vein wall. The user can command the handle 172 to deliver energy (e.g., electrical, direct current, alternating current, radio frequency, non-radio frequency) to the expandable member 440 to ablate the vein wall, which can damage the vein wall. In some variants, energy can be supplied by one or more power sources (e.g., battery, generator, power connection, wall outlet, etc.). In some variants, direct current can resistively heat the expandable member 440 to damage (e.g., ablate) the vein wall. In some variants, the handle 172 can deliver energy to the expandable member 410 in pulses. In some variants, the handle 172 and/or a device in communication with the handle 172 can automatically adjust energy delivery based on temperatures sensed by the temperature sensor 330. In some variants, the expandable member 440 can be moved (e.g., rotated and/or translated within the vein), automatically and/or manually, to mechanically damage the vein wall. For example, in some variants, the user can move the handle 172 distally and/or proximally to translate the expandable member 440 within the vein to damage the vein wall. The ablation assembly 439 can be proximally retracted along a length of the vein to damage the length of the vein. The damage to the vein wall can cause the vein to collapse, which can close off the vein and ultimately result in the vein being reabsorbed into the body. With ablation finished at the target site, the expandable member 440 can be collapsed for removal of the ablation assembly 439. For example, after overcoming the biasing force of the one or more levers 464, the dial 446 can be rotated in the second direction to rotate the shaft 424 to collapse the expandable member 440 inward. With the expandable member 440 collapsed, the ablation assembly 439 can be proximally retracted out of the vasculature of the patient. In some variants, the expandable member 440 can be covered (e.g., sheathed) prior to removal.

The expandable members (e.g., heating elements) described herein can have a higher resistance than the conductors (e.g., conductors 124, 126, 321, and/or 323) that deliver energy to the expandable members to facilitate heating. In some variants, the expandable members can have a higher resistance compared to the conductors 124, 126, 321, and/or 323 due to material and/or geometric characteristics. For example, the expandable members can include a material with a higher resistance than the conductors 124, 126, 321, and/or 323 such as nickel titanium (e.g., Nitinol). The conductors 124, 126, 321, and/or 323 can include a material with lower resistance (e.g., copper, gold, aluminum, steel, silver, brass, platinum, iron, etc.). The expandable members (e.g., conductors thereof) can include a smaller cross-sectional size than the conductors 124, 126, 321, and/or 323 for increased resistance. In some variants, the conductors 124, 126, 321, and/or 323 and/or expandable members can include the same or different materials. In some variants, the conductors 124, 126, 321, and/or 323 can include the same or different materials. For example, the conductors 124, 321 to deliver electrical energy to the expandable members can include a material with lower resistance, such as copper, to avoid losses while the conductors 126, 323 can include a less expensive material, such as aluminum, to conduct electrical energy away from the expandable member when losses may be less important. In some variants, the conductors 124, 126, 321, and/or 323 can include the same material, which can at least include any of the foregoing. In some variants, the expandable members can include the same material as the conductors 124, 126, 321, and/or 323 (e.g., the material can be continuous between the conductors 124, 126, 321, and/or 323 and/or expandable member) but the geometric characteristics (e.g., cross-sectional size) can be varied (e.g., cross-sectional size reduced) to increase resistance to facilitate sufficient resistive heating. In some variants, the expandable members can be coupled (e.g., welded, clamped, bonded, clinched, fused, crimped) to the conductor 124 and/or conductor 126.

The heated elements described herein can be expandable members. The expandable members described herein can be heated elements. The expandable members can be self-expanding and/or manually expanded. In some variants, damaging a wall of a blood vessel with thermal energy can be referred to as ablation herein. In some variants, the heating elements and/or expandable member can be heated to less than 120, 110, 100, 90, 80, 70, 60, 50, or 40 degrees Celsius.

The endovenous devices described herein can use direct current, alternating current, pulse width modulation, radio frequency, non-radio frequency, and/or electric field to thermally damage (e.g., ablate) a wall of a blood vessel (e.g., vein). Utilizing direct current can provide the advantages described herein. The thermal ablation techniques described herein can be accompanied by mechanical techniques to damage the vein wall (e.g., endothelium). The endovenous devices described herein can employ low power to ablate, which can, in some variants, allow a battery to be used as a power supply.

As described herein, the endovenous devices can include temperature sensors disposed proximate the expandable members to sense temperature, which can provide for closed loop control of the energy delivery to the expandable members. The endoveous devices described herein can employ pulse-width modulation of the energy delivery to control heat. The handles described herein can include controllers to automatically adjust energy delivery based on temperature.

In some variants, the devices, such as the endovenous devices described herein, can be used to treat blood vessels, such as an artery and/or a vein (e.g., varicose vein).

The expanding and self-expanding features described herein can expand to a variety of outer diameter sizes, which can include between 0.1 to 15 millimeters and/or 1 to 15 millimeters. In some variants, the expanding and self-expanding features prior to expansion can have a diameter less than 1 millimeter. In some variants, the sheaths (e.g., delivery sheaths) described herein that can be disposed over the elongate element can be 1 Fr-10 Fr and/or 0.33 millimeters to 3.33 millimeters.

The heating elements described herein can be heated to a variety of temperatures, which can at least include 0-150 degrees Celsius or 60-120 degrees Celsius. The heating elements described herein can include different lengths, which can at least include 0-50 centimeters or 1-10 centimeters. The power sources disclosed herein, such as the battery, can have a power range of 0-1000 watts or 10-80 watts or others.

In some variants, the endovenous devices described herein can utilize alternating current. In some variants, the endovenous devices described herein can include a switch to alternate the direction of current flow for different frequencies for biphasic.

The endovenous devices (e.g., ablation assemblies) described herein can be used to treat any venous disease (e.g., chronic venous disease), not just varicose veins, where closure is desired. For example, the endovenous devices (e.g., ablation assemblies) described herein can be used to at least treat veins with a Chronic Venous Disease Evaluation and Classification (CEAP) score of C1-C3. Any description related to treating varicose veins herein can be applied to treating any vein with a venous disease.

### Certain Examples

Below is a list of non-limiting examples described herein. These examples are for illustrative purposes and should not be viewed to restrict or limit the disclosure herein in any way.

In a 1st Example, an endovenous device for treating a vein, the endovenous device comprising: an elongate element comprising a first conductor, a second conductor, and a self-expanding heating element, the self-expanding heating element disposed at a distal portion of the elongate element; and a handle coupled to the elongate element, the handle comprising a battery; wherein the elongate element is configured to be disposed through vasculature to place the self-expanding heating element within the vein; wherein the self-expanding heating element is configured to self-expand to contact an inner wall of the vein; wherein the battery is configured to deliver direct current through the first conductor to the self-expanding heating element to raise a temperature of the self-expanding heating element with resistive heating; and wherein the second conductor is configured to conduct direct current away from the self-expanding heating element.

In a 2nd Example, the endovenous device of Example 1, wherein the elongate element is a catheter.

In a 3rd Example, the endovenous device of any of the preceding Examples, wherein the elongate element comprises a thermocouple proximate the self-expanding heating element.

In a 4th Example, the endovenous device of Example 3, wherein the handle comprises a controller configured to control the direct current flowing from the battery to the self-expanding heating element based at least in part on temperature sensed by the thermocouple.

In a 5th Example, the endovenous device of any of the preceding Examples, wherein the self-expanding heating element comprises a loop shape when self-expanded.

In a 6th Example, the endovenous device of any of the preceding Examples, wherein the self-expanding heating element comprises a coil shape when self-expanded.

In a 7th Example, the endovenous device of any of the preceding Examples, wherein the self-expanding heating element comprises a ring shape when self-expanded.

In an 8th Example, the endovenous device of any of the preceding Examples, wherein the self-expanding heating element comprises a plurality of leads that bow outward relative to the elongate element when self-expanded.

In a 9th Example, the endovenous device of any of the preceding Examples, further comprising a sheath disposed over at least a portion of the elongate element, the sheath configured to thermally insulate.

In a 10th Example, the endovenous device of Example 9, wherein an annular gap is disposed between the sheath and the elongate element, the annular gap configure to deliver one or more substances to inside the vein.

In a 11th Example, the endovenous device of Example 10, wherein the one or more substances comprises a local anesthetic.

In a 12th Example, the endovenous device of any of the preceding Examples, wherein the first conductor and the second conductor are electrically insulated.

In a 13th Example, the endovenous device of any of the preceding Examples, wherein the elongate element comprises insulation to electrically insulate the first conductor and the second conductor.

In a 14th Example, the endovenous device of any of the preceding Examples, wherein the self-expanding heating element is coupled to the first conductor and the second conductor.

In a 15th Example, the endovenous device of any of the preceding Examples, wherein the self-expanding heating element comprises a material that is more electrically resistive than that of the first conductor and the second conductor.

In a 16th Example, the endovenous device of any of the preceding Examples, wherein the self-expanding heating element comprises nickel titanium.

In a 17th Example, the endovenous device of any of the preceding Examples, wherein at least one of the first conductor and the second conductor comprise copper.

In an 18th Example, the endovenous device of any of the preceding Examples, wherein at least one of the first conductor and the second conductor comprise gold.

In a 19th Example, the endovenous device of any of the preceding Examples, wherein the self-expanding heating element comprises a geometry that is more electrically resistive than that of the first conductor and the second conductor.

In a 20th Example, the endovenous device of any of the preceding Examples, wherein self-expanding heating element comprises a cross-sectional size that is smaller than that of the first conductor and the second conductor.

In a 21st Example, the endovenous device of any of the preceding Examples, wherein self-expanding heating element comprises a wire.

In a 22nd Example, the endovenous device of any of the preceding Examples, wherein the battery is rechargeable.

In a 23rd Example, the endovenous device of any of the preceding Examples, wherein the elongate element comprises a lumen configured to receive one or more devices therethrough.

In a 24th Example, the endovenous device of any of the preceding Examples, wherein the elongate element is steerable.

In a 25th Example, the endovenous device of any of the preceding Examples, wherein the elongate element comprises a first connector and the handle comprises a second connector, and wherein the first connector and the second connector are configured to interface to couple the handle and the elongate element.

In a 26th Example, the endovenous device of Example 25, wherein the first connector and the second connector are multi-pin connectors.

In a 26th Example, the endovenous device of Example 25, wherein the handle comprises a button to enable a user to control delivering direct current to the self-expanding heating element.

In a 28th Example, the endovenous device of any of the preceding Examples, wherein the handle comprises a buzzer.

In a 29th Example, the endovenous device of any of the preceding Examples, wherein the handle comprises an indicator light.

In a 30th Example, the endovenous device of any of the preceding Examples, wherein the handle comprises a housing with an interior to house internal components.

In a 31st Example, the endovenous device of Example 30, wherein the handle comprises a chassis configured to receive the internal components thereon prior to insertion into the interior of the housing.

In a 32nd Example, the endovenous device of any of the preceding Examples, wherein the self-expanding heating element comprises abrasive features configured to contact the inner wall of the vein.

In a 33rd Example, the endovenous device of any of the preceding Examples, wherein the self-expanding heating element is configured to move to disrupt endothelium of the inner wall.

In a 34th Example, the endovenous device of any of the preceding Examples, wherein the self-expanding heating element is configured to self-expand to a size based on an inner diameter of the inner wall of the vein.

In a 35th Example, the endovenous device of any of the preceding Examples, further comprising a controller to switch a direction of flow of the direct current through the first conductor, second conductor, and self-expanding heating element for variable frequency.

In a 36th Example, the endovenous device of Example 35, wherein the variable frequency is between 3 kHz and 300 GHz.

In a 37th Example, the endovenous device of any of the preceding Examples, wherein the self-expanding heating element self-expands up to fifteen millimeters.

In a 38th Example, the endovenous device of any of the preceding Examples, wherein heating element is heated to 0-150 degrees Celsius.

In a 39th Example, the endovenous device of any of the preceding Examples, wherein heating element is heated to 60-120 degrees Celsius.

In a 40th Example, the endovenous device of any of the preceding Examples, wherein a power of the battery is 0-1000 W.

In a 41st Example, the endovenous device of any of the preceding Examples, wherein a power of the battery is 10-80 W.

In a 42nd Example, the endovenous device of any of the preceding Examples, wherein the self-expanding heating element comprises an outer diameter of less than 1 millimeter prior to self-expansion.

In a 43rd Example, an endovenous device for treating a vein, the endovenous device comprising: an elongate element comprising a first conductor, a second conductor, and a self-expanding heating element, the self-expanding heating element disposed at a distal portion of the elongate element; and a handle coupled to the elongate element; wherein the elongate element is configured to be disposed through vasculature to place the self-expanding heating element within the vein; wherein the self-expanding heating element is configured to self-expand to contact an inner wall of the vein; wherein the first conductor is configured to conduct direct current to the self-expanding heating element to raise a temperature of the self-expanding heating element with resistive heating; and wherein the second conductor is configured to conduct direct current away from the self-expanding heating element.

In a 44th Example, the endovenous device of Example 43, wherein the handle is configured to be coupled to an external power source for electrical energy.

In a 45th Example, the endovenous device of Example 43 or 44, further comprising any of the features of Examples 1-42.

In a 46th Example, an endovenous device for treating a vein, the endovenous device comprising: an elongate element comprising a first conductor, a second conductor, and a heating element, the heating element disposed at a distal portion of the elongate element; and a handle coupled to the elongate element; wherein the elongate element is configured to be disposed through vasculature to place the heating element within the vein; wherein the first conductor is configured to conduct direct current to the heating element to raise a temperature of the heating element with resistive heating; and wherein the second conductor is configured to conduct direct current away from the heating element.

In a 47th Example, the endovenous device of Example 46, wherein the heating element is configured to expand to contact an inner wall of the vein.

In a 48th Example, the endovenous device of Example 46 or 47, wherein the handle comprises a battery.

In a 49th Example, the endovenous device of any of Examples 46-48, further comprising a self-expanding feature configured to expand to contact an inner wall of the vein.

In a 50th Example, the endovenous device of Example 49, wherein the self-expanding feature is configured to move to disrupt the inner wall of the vein.

In a 51st Example, a method of treating occluding a vein, the method comprising: percutaneously accessing vasculature; distally advancing an elongate element of an endovenous device through the vasculature to place a heating element within the vein; delivering direct current to the heating element to raise a temperature of the heating element with resistive heating; proximally retracting the elongate element along a length of the vein to occlude the vein; and removing the elongate element from the vasculature.

In a 52nd Example, the endovenous device of Example 51, further comprising expanding the heating element to contact an inner wall of the vein.

In a 53rd Example, the endovenous device of Example 52, wherein the heating element self-expands.

In a 54th Example, the endovenous device of Examples 51-53, further comprising delivering an agent within the vein.

In a 55th Example, the endovenous device of Example 54, wherein the agent is delivered through a gap between the elongate element and a sheath.

In a 56th Example, the endovenous device of Example 54 or 55, wherein the agent is a local anesthetic.

In a 57th Example, an endovenous device for treating a vein, the endovenous device comprising: an elongate element comprising a first conductor, a second conductor, and a self-expanding heating element, the self-expanding heating element disposed at a distal portion of the elongate element; and a handle coupled to the elongate element; wherein the elongate element is configured to be disposed through vasculature to place the self-expanding heating element within the vein; wherein the self-expanding heating element is configured to self-expand to contact an inner wall of the vein; wherein the self-expanding heating element is configured to move to disrupt the inner wall of the vein; wherein the first conductor is configured to conduct direct current to the self-expanding heating element to raise a temperature of the self-expanding heating element with resistive heating; and wherein the second conductor is configured to conduct direct current away from the self-expanding heating element.

In a 58th Example, the endovenous device of Example 57, wherein the self-expanding heating element is configured to rotate to disrupt the inner wall of the vein.

In a 59th Example, the endovenous device of Example 57, wherein the self-expanding heating element is configured to oscillate to disrupt the inner wall of the vein.

In a 60th Example, the endovenous device of Example 57, wherein the self-expanding heating element is configured to vibrate to disrupt the inner wall of the vein.

In a 61 st Example, an endovenous device for treating a vein, the endovenous device comprising: an elongate element comprising a first conductor, a second conductor, and a self-expanding heating element, the self-expanding heating element disposed at a distal portion of the elongate element; a sheath configured to be disposed over the elongate element; and a handle coupled to the elongate element; wherein the elongate element is configured to be disposed through vasculature to place the self-expanding heating element within the vein; wherein the sheath is configured to deliver a substance to within the vein; wherein the self-expanding heating element is configured to self-expand to contact an inner wall of the vein; wherein the first conductor is configured to conduct direct current to the self-expanding heating element to raise a temperature of the self-expanding heating element with resistive heating; and wherein the second conductor is configured to conduct direct current away from the self-expanding heating element.

In a 62nd Example, the endovenous device of Example 61, wherein the substance is a local anesthetic for numbing.

In a 63rd Example, the endovenous device of Example 61 or 62, wherein the sheath comprises a distal opening through which the substance is configured to be delivered.

In a 64th Example, the endovenous device of any of Examples 61-63, wherein the sheath comprises one or more openings through a peripheral wall through which the substance is configured to be delivered.

In a 65th Example, an endovenous device for treating a vein, the endovenous device comprising: an elongate element comprising a first conductor, a second conductor, and a self-expanding heating element, the self-expanding heating element disposed at a distal portion of the elongate element; and a handle coupled to the elongate element; wherein the elongate element is configured to be disposed through vasculature to place the self-expanding heating element within the vein; wherein the self-expanding heating element is configured to self-expand to contact an inner wall of the vein; wherein the first conductor is configured to conduct direct current to the self-expanding heating element to raise a temperature of the self-expanding heating element with resistive heating; and wherein the second conductor is configured to conduct direct current away from the self-expanding heating element.

In a 66th Example, an endovenous device for treating a vein, the endovenous device comprising: an elongate element comprising a first conductor, a second conductor, a thermocouple, and a self-expanding heating element, the self-expanding heating element disposed at a distal portion of the elongate element; and a handle coupled to the elongate element, the handle comprising a battery and a controller; wherein the elongate element is configured to be disposed through vasculature to place the self-expanding heating element within the vein; wherein the self-expanding heating element is configured to self-expand to contact an inner wall of the vein; wherein the battery is configured to deliver direct current through the first conductor to the self-expanding heating element to raise a temperature of the self-expanding heating element with resistive heating and the second conductor is configured to conduct direct current away from the self-expanding heating element; wherein the thermocouple is configured to sense temperature proximate the self-expanding heating element; and wherein the controller is configured to control delivery of direct current by the battery based on temperature sensed by the thermocouple.

In a 67th Example, the endovenous device of Example 66, wherein the controller is configured to pulse delivery of direct current by the battery.

In a 68th Example, the endovenous device of Example 66 or 67, wherein the battery is configured to heat the self-expanding heating element to low temperatures.

In a 69th Example, the endovenous device of Example 68, wherein the low temperatures comprises less than 120 degrees Celsius.

In a 70th Example, the endovenous device of Example 68, wherein the low temperatures comprises less than 100 degrees Celsius.

In a 71st Example, the endovenous device of Example 68, wherein the low temperatures comprises less than 80 degrees Celsius.

In a 72nd Example, the endovenous device of Example 68, wherein the low temperatures comprises less than 60 degrees Celsius.

In a 73rd Example, the endovenous device of Example 68, wherein the low temperatures comprises less than 40 degrees Celsius.

In a 74th Example, an endovenous device for treating a blood vessel, the endovenous device comprising: an elongate element comprising a first conductor, a second conductor, and a heating element, the first conductor configured to deliver energy to the heating element to raise a temperature of the heating element to damage a wall of the blood vessel, and the second conductor configured to conduct energy away from the heating element.

In a 75th Example, the endovenous device of Example 74, wherein the first conductor is configured to deliver energy from a battery to the heating element.

In a 76th Example, the endovenous device of Example 75, wherein the elongate element is configured to couple with a handle comprising a battery to deliver energy to the first conductor.

In a 77th Example, the endovenous device of Example 74 or 75, further comprising a handle with a battery, wherein the elongate element is configured to be coupled to the handle.

In a 78th Example, the endovenous device of any of Examples 74-77, wherein the energy comprises direct current.

In a 79th Example, the endovenous device of any of Examples 74-78, wherein the elongate element further comprises a temperature sensor disposed proximate the heating element.

In an 80th Example, the endovenous device of Example 79, further comprising a controller to control energy delivered to the heating element based on temperatures sensed by the temperature sensor.

In an 81st Example, the endovenous device of Example 80, wherein the controller utilizes pulse-width modulation to control energy delivered to the heating element.

In an 82nd Example, the endovenous device of any of Examples 74-81, wherein the heating element comprises a material that is more electrically resistive than that of the first conductor.

In an 83rd Example, the endovenous device of any of Examples 74-82, wherein the heating element comprises a geometry that is more electrically resistive than that of the first conductor.

In an 84th Example, the endovenous device of any of Examples 74-83, wherein the heating element comprises a conductor having a cross-sectional size that is small than that of the first conductor.

In an 85th Example, the endovenous device of any of Examples 74-84, further comprising an outer tube configured to be advanced to cover the heated element and retracted to uncover the heated element.

In an 86th Example, the endovenous device of any of Examples 74-85, wherein the heated element is configured to expand to contact a wall of the blood vessel.

In an 87th Example, the endovenous device of Example 86, wherein the heated element is configured to self-expand.

In an 88th Example, the endovenous device of Example 86, wherein the heated element is configured to expand to a coil shape.

In an 89th Example, the endovenous device of Example 86, wherein the heated element is configured to expand to a double coil shape with a first portion carrying energy distally and a second portion carrying energy proximally.

In an 90th Example, the endovenous device of Example 88 or 89, further comprising an outer tube and an actuator, the actuator comprising a housing and a slider assembly with a lock, the slider assembly coupled to the outer tube, wherein the lock is configured to be rotated in a first direction to disengage from the housing to distally advance to position the outer tube over the heated element, and wherein the lock is configured to be rotated in a second direction to engage with the housing to impede distal advancement of the slider assembly and the outer tube.

In a 91st Example, the endovenous device of Example 86, wherein the heated element is configured to expand to a loop.

In a 92nd Example, the endovenous device of Example 86, wherein the heated element is configured to expand to a fusiform shape.

In a 93rd Example, the endovenous device of Example 92, further comprising a shaft and an actuator, the shaft coupled to a distal portion of the heated element, and the actuator comprising a dial configured to be rotated in a first direction to advance the shaft to collapse the heated element and in a second direction to retract the shaft to expand the heated element.

In a 94th Example, the endovenous device of Example 86, wherein the heated element is configured to expand to a cone shape.

In a 95th Example, the endovenous device of Example 94, further comprising a shaft and an actuator, the shaft coupled to a distal portion of the heated element, and the actuator comprising a dial configured to be rotated in a first direction to rotate the shaft to expand the heated element and in a second direction to rotate the shaft to collapse the heated element.

In a 96th Example, an endovenous device for treating a blood vessel, the endovenous device comprising: an elongate element comprising an expandable member, a conductor, and a temperature sensor, the expandable member configured to expand to contact a wall of the blood vessel, and the conductor configured to deliver energy to the expandable member to damage the wall of the blood vessel.

In a 97th Example, the endovenous device of Example 96, wherein the energy comprises direct current that resistively heats the expandable member to damage the wall of the blood vessel with thermal energy.

In a 98th Example, the endovenous device of Example 96, wherein the energy is alternating current.

In a 99th Example, the endovenous device of Example 96, wherein the energy comprises radio frequency.

In a 100th Example, the endovenous device of any of Examples 96-99, further comprising a controller to control energy delivered to the expandable member based on temperatures sensed by the temperature sensor.

In a 101st Example, the endovenous device of Example 100, wherein the controller utilizes pulse-width modulation to control energy delivered to the expandable member.

In a 102nd Example, the endovenous device of any of Examples 96-101, wherein the expandable member is configured to self-expand.

In a 103rd Example, the endovenous device of any of Examples 96-102, wherein the expandable member is configured to expand to a coil shape.

In a 104th Example, the endovenous device of any of Examples 96-102, wherein the expandable member is configured to expand to a double coil shape with a first portion carrying energy distally and a second portion carrying energy proximally.

In a 105th Example, the endovenous device of Example 103 or 104, further comprising an outer tube and an actuator, the actuator comprising a housing and a slider assembly with a lock, the slider assembly coupled to the outer tube, wherein the lock is configured to be rotated in a first direction to disengage from the housing to distally advance to position the outer tube over the expandable member, and wherein the lock is configured to be rotated in a second direction to engage with the housing to impede distal advancement of the slider assembly and the outer tube.

In a 106th Example, the endovenous device of any of Examples 96-102, wherein the expandable member is configured to expand to a loop.

In a 107th Example, the endovenous device of any of Examples 96-102, wherein the expandable member is configured to expand to a fusiform shape.

In a 108th Example, the endovenous device of Example 107, further comprising a shaft and an actuator, the shaft coupled to a distal portion of the expandable member, and the actuator comprising a dial configured to be rotated in a first direction to advance the shaft to collapse the expandable member and in a second direction to retract the shaft to expand the expandable member.

In a 109th Example, the endovenous device of any of Examples 96-102, wherein the expandable member is configured to expand to a cone shape.

In a 110th Example, the endovenous device of Example 109, further comprising a shaft and an actuator, the shaft coupled to a distal portion of the expandable member, and the actuator comprising a dial configured to be rotated in a first direction to rotate the shaft to expand the expandable member and in a second direction to rotate the shaft to collapse the expandable member.

In a 111th Example, a method of treating a blood vessel, the method comprising: percutaneously accessing vasculature; distally advancing an elongate element of an endovenous device through the vasculature to place a heating element within the blood vessel; expanding the heating element to contact an inner wall of the blood vessel; delivering energy to the heating element to damage the inner wall of the blood vessel with thermal energy; proximally retracting the elongate element along a length of the blood vessel to damage the length of the inner wall of the blood vessel; and removing the elongate element from the vasculature.

In a 112th Example, the endovenous device of Example 111, wherein delivering energy comprises delivering direct current from a battery to resistively heat the heating element.

### Terminology

Although the systems and methods have been disclosed in the context of certain embodiments and examples, it will be understood by those skilled in the art that the systems and methods extend beyond the specifically disclosed embodiments to other alternative embodiments and/or uses of the embodiments and certain modifications and equivalents thereof. Various features and aspects of the disclosed embodiments can be combined with or substituted for one another in order to form varying modes. The scope of this disclosure should not be limited by the particular disclosed embodiments described herein.

Methods of using the foregoing system(s) (including device(s), apparatus(es), assembly(ies), structure(s) or the like) are included; the methods of use can include using or assembling any one or more of the features disclosed herein to achieve functions and/or features of the system(s) as discussed in this disclosure. Methods of manufacturing the foregoing system(s) are included; the methods of manufacture can include providing, making, connecting, assembling, and/or installing any one or more of the features of the system(s) disclosed herein to achieve functions and/or features of the system(s) as discussed in this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Some embodiments have been described in connection with the accompanying drawings. Components can be added, removed, and/or rearranged. Orientation references such as, for example, “top” and “bottom” are for ease of ease of discussion and may be rearranged such that top features are proximate the bottom and bottom features are proximate the top. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In summary, various embodiments and examples of juicing devices and methods have been disclosed. Although the systems and methods have been disclosed in the context of those embodiments and examples, it will be understood by those skilled in the art that this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims that follow.

What is claimed is:

1. An endovenous device for treating a blood vessel, the endovenous device comprising:
an elongate element comprising a first conductor, a second conductor, and a heating element, the first conductor configured to deliver energy to the heating element to raise a temperature of the heating element to damage a wall of the blood vessel, and the second conductor configured to conduct energy away from the heating element.

2. The endovenous device of claim 1, wherein the first conductor is configured to deliver energy from a battery to the heating element.

3. The endovenous device of claim 1, further comprising a handle with a battery, wherein the elongate element is configured to be coupled to the handle.

4. The endovenous device of claim 1, wherein the energy comprises direct current to resistively heat the heating element.

5. The endovenous device of claim 1, wherein the elongate element further comprises a temperature sensor disposed proximate the heating element.

6. The endovenous device of claim 5, further comprising a controller to control energy delivered to the heating element based on temperatures sensed by the temperature sensor.

7. The endovenous device of claim 6, wherein the controller utilizes pulse-width modulation to control energy delivered to the heating element.

8. The endovenous device of claim 1, wherein the heating element comprises a material that is more electrically resistive than that of the first conductor.

9. The endovenous device of claim 1, wherein the heating element comprises a geometry that is more electrically resistive than that of the first conductor.

10. The endovenous device of claim 1, wherein the heating element comprises a conductor having a cross-sectional size that is small than that of the first conductor.

11. The endovenous device of claim 1, further comprising an outer tube configured to be advanced to cover the heating element and retracted to uncover the heating element.

12. The endovenous device of claim 1, wherein the heating element is configured to expand to contact a wall of the blood vessel.

13. The endovenous device of claim 12, wherein the heating element is configured to self-expand.

14. An endovenous device for treating a blood vessel, the endovenous device comprising:
an elongate element comprising an expandable member, a conductor, and a temperature sensor, the expandable member configured to expand to contact a wall of the blood vessel, and the conductor configured to deliver energy to the expandable member to damage the wall of the blood vessel.

15. The endovenous device of claim 14, wherein the energy comprises direct current that resistively heats the expandable member to damage the wall of the blood vessel with thermal energy.

16. The endovenous device of claim 14, further comprising a controller to control energy delivered to the expandable member based on temperatures sensed by the temperature sensor.

17. The endovenous device of claim 16, wherein the controller utilizes pulse-width modulation to control energy delivered to the expandable member.

18. The endovenous device of claim 14, wherein the expandable member is configured to self-expand.

19. A method of treating a blood vessel, the method comprising:
percutaneously accessing vasculature;
distally advancing an elongate element of an endovenous device through the vasculature to place a heating element within the blood vessel;
expanding the heating element to contact an inner wall of the blood vessel;
delivering energy to the heating element to damage the inner wall of the blood vessel with thermal energy;
proximally retracting the elongate element along a length of the blood vessel to damage the length of the inner wall of the blood vessel; and
removing the elongate element from the vasculature.

20. The method of claim 19, wherein delivering energy comprises delivering direct current from a battery to resistively heat the heating element.
